(19) United States
(12) Patent Application Publication
(10) Pub. No.: US 2025/0367132 A1
(43) Pub. Date: Dec. 4, 2025

(54) SOLID LIPID NANOPARTICLES FOR ENCAPSULATION AND DELIVERY OF BIOACTIVE COMPOUNDS AND METHODS OF MAKING THE SAME

(71) Applicants: Chris Baek, Des Plaines, IL (US); Matthew Gdovin, Spring Branch, TX (US); Kevin Ita, American Canyon, CA (US)

(72) Inventors: Chris Baek, Des Plaines, IL (US); Matthew Gdovin, Spring Branch, TX (US); Kevin Ita, American Canyon, CA (US)

(73) Assignee: Yubeck, Inc., Des Plaines, IL (US)

(21) Appl. No.: 19/298,695

(22) Filed: Aug. 13, 2025

Related U.S. Application Data
(63) Continuation of application No. 19/093,251, filed on Mar. 28, 2025.
(60) Provisional application No. 63/573,355, filed on Apr. 2, 2024.

(51) Int. Cl. A61K 9/51; A61K 9/19; A61K 31/352; A61K 31/4045; A61K 31/675; A61K 35/748; A61K 36/06; A61K 38/16; B82Y 5/00

(52) U.S. Cl. CPC A61K 9/5123; A61K 9/19; A61K 31/352; A61K 31/4045; A61K 31/675; A61K 35/748; A61K 36/06; A61K 38/164; B82Y 5/00

(57) ABSTRACT

Solid lipid nanoparticles (SLNs) for delivery of bioactive compounds are disclosed. The SLNs comprise a lipid matrix and surfactant layer encapsulating at least one bioactive compound selected from vitamins, minerals, enzymes, algae-derived bioactives, proteins, peptides, amino acids, antioxidants, small synthetic molecules, plant-derived volatile compounds, or botanical extracts. The SLNs exhibit submicron particle size, low polydispersity, and sufficient surface charge to ensure colloidal stability and efficient delivery. In one embodiment, algae-based bioactives, such as phycocyanin or fucoxanthin, are encapsulated using only natural and sustainable lipids and surfactants to improve bioavailability and support environmentally friendly formulations. The SLNs may be formulated for oral, topical, transdermal, injectable, ophthalmic, mucosal, textile, veterinary, or agricultural administration. Applications include human and animal health, functional foods, skincare, nutrient supplementation, and crop treatment. The disclosed SLNs offer a biocompatible and scalable delivery system that protects sensitive compounds, enables sustained release, and enhances absorption across diverse industries.

### CROSS-REFERENCE TO RELATED APPLICATIONS

The present application is a continuation application of application Ser. No. 19/093,251, filed Mar. 28, 2025, which takes priority from Provisional App. No. 63/573,355, filed Apr. 2, 2024, which are incorporated herein by reference.

### BACKGROUND

### Field of the Invention

The present invention relates to the field of nanoencapsulation and drug delivery systems, specifically to solid lipid nanoparticles (SLNs) for the encapsulation and delivery of bioactive compounds, including vitamins, minerals, enzyme, algae-derived material, proteins, peptides, amino acids, antioxidant, synthetic small molecule, plant-derived volatile compounds, or naturally derived phytochemical compounds, such as botanical extracts and polyphenols. This invention specifically addresses the composition, methods, and application of SLNs for improving the bioavailability, stability, and controlled release of a wide range of bioactive ingredients across pharmaceutical, nutraceutical, dermatologic, cosmetic, veterinary, textile, food and beverage, and agricultural applications. The SLNs described herein may be formulated for oral, topical, transdermal, injectable, mucosal, ophthalmic, and other delivery routes. Additionally, the use of SLNs can be used independently or incorporated into secondary delivery systems, such as dissolvable microneedles (DMNs).

### Background

Many bioactive compounds, such as vitamins, minerals, proteins, peptides, amino acids, polyphenols, small synthetic compounds, and algae-derived bioactives, such as Spirulina and phycocyanin, face significant formulation challenges that hinder their efficacy. These challenges include poor solubility, enzymatic degradation, low bioavailability, and instability due to environmental factors such as heat, light, and oxidation. Conventional oral and injectable formulations often fail to address these issues, resulting in reduced therapeutic benefits, variable absorption rates, and the need for higher or more frequent dosing to achieve desired effects. Highly pigmented bioactives, such as phycocyanin and Spirulina, present aesthetic and formulation challenges in applications, such as cosmetics and functional foods, where color neutrality is often desired.

Certain hydrophobic compounds exhibit poor water solubility, leading to inefficient absorption in the gastrointestinal (GI) tract. For example, compounds like omega-3 fatty acids, vitamin D3, and curcumin suffer from poor solubility, limiting their effectiveness in traditional formulations. Additionally, micronutrients such as iron, magnesium, and vitamin B12, are poorly absorbed due to low solubility, competitive dietary interactions, or impaired update in certain populations.

Large-molecule proteins, such as therapeutic proteins and functional peptides are difficult to deliver orally and transdermally due to their molecular size, instability, and limited permeability through biological membranes. Spirulina-derived compounds, although rich in nutritional and therapeutic potential, present additional formulation challenges in food and nutraceutical applications due to their intense green pigmentation, odor, and chemical instability. Phycocyanin, a key bioactive protein-pigment found in Spirulina, is highly sensitive to degradation from light, heat, pH, and oxidation and is difficult to deliver effectively, limiting its use in pharmaceutical, biomedical, and dermatological applications. Without effective stabilization and delivery strategies, maintaining phycocyanin's bioactivity and ensuring its efficient absorption remain significant challenges, necessitating advanced encapsulation and/or transdermal delivery technologies.

There remains an unmet need for a delivery platform capable of stabilizing sensitive bioactives, improving solubility and absorption, and enabling controlled or sustained release as well as addressing sensory challenges such as color and odor in natural compounds. A system that supports flexible routes of administration, including oral, topical, transdermal, mucosal, and food-based delivery, would provide a significant advance over current technologies.

### SUMMARY OF THE INVENTION

An object of the present invention is to provide a solid lipid nanoparticle with a bioactive compound encapsulated within the lipid.

Another object of the present invention is to provide a composition comprising a bioactive compound encapsulated within a solid lipid nanoparticle, in a way that makes the bioactive compound easier to absorb.

Another object of the present invention is to provide a composition comprising a bioactive compound encapsulated within a solid lipid nanoparticle that can be absorbed transdermally via a dissolvable microneedle system.

According to an aspect of the present invention, a solid lipid nanoparticle (SLN) is provided, comprising a generally spherical particle comprising a lipid matrix with at least one bioactive compound dispersed within the lipid matrix. A surfactant layer forms a coating on the exterior of the particle. The particle has a diameter of 10 nm to approximately 1000 nm, a polydispersity index (PDI) of less than 0.30, and a zeta potential (absolute value) of at least 25 mV.

In an embodiment, the bioactive compound could be a vitamin, mineral, enzyme, algae-derived material, protein, peptide, amino acid, antioxidant, synthetic small molecule, a botanical extract, plant derived volatile compound, a polyphenol, or a naturally derived phytochemical compound. In an embodiment, the bioactive compound could be derived from algae. The bioactive compound could be whole Spirulina or C-Phycocyanin. There could be more than one bioactive compound embedded in the same SLN.

The lipid matrix could comprise multiple lipids. In an embodiment, all the lipids are natural. Similarly, the surfactant layer could comprise two or more distinct surfactants, and in an embodiment, the surfactant or surfactants could all be natural.

According to an aspect of the invention, a method of making a solid lipid nanoparticle composition is provided. To make the solid lipid nanoparticle composition, a bioactive compound is dissolved in a solvent (distilled water, ethanol, or pharmaceutically acceptable solvent) to create a bioactive phase; a lipid is dissolved in another solvent to create a lipid phase (the solvent could be ethanol or a pharmaceutically acceptable solvent). The lipid phase is heated to above the melting point of the lipid, and the lipid phase and bioactive phase are mixed, and a surfactant solution is added to the mixture. The bioactive compound could be mixed with the lipid first, or with the surfactant first, depending on the compound. The mixture is heated and sonicated to form SLNs and then cooled in an aqueous phase or dried.

In an embodiment, the bioactive compound is sonicated before mixing the bioactive phase with the lipid phase. This sonication is done to reduce the particle size of the bioactive compound to make it possible to encapsulate it in SLNs with a desired diameter of <350 nm. The bioactive phase is sonicated until its mean particle size is 100 nm or less.

According to an aspect of the present invention, a system is provided for delivering a bioactive compound to a living being. This delivery system could be done by a transdermal delivery system comprising at least one dissolvable microneedle, a topical formulation, a food item, an ophthalmic drop, an oral medication, a nasal spray, an inhaler, a suppository, or a wearable product, each of which comprise at least one SLN.

### LIST OF FIGURES

FIG. 1 shows a view of a solid lipid nanoparticle according to an embodiment of the present invention.

FIG. 2 shows various characteristics of the SLNs produced by the methods of an embodiment of the present invention.

FIG. 3 shows a diagram of a process of making solid lipid nanoparticles containing vitamin D according to an embodiment of the present invention.

FIG. 4 shows a diagram of the impact of various cooling methods on SLN parameters.

FIG. 5 shows a diagram of a process of making SLNs containing Spirulina according to an embodiment of the present invention.

FIG. 6 shows a diagram of the impact of sonication on Spirulina particle size.

### DETAILED DESCRIPTION

### Overview

In one aspect of the present invention, solid lipid nanoparticles (SLNs) are disclosed. SLNs are submicron-sized particles with a diameter ranging from 10 nm to 1000 nm, preferably less than 350 nm, composed of biocompatible lipids stabilized by surfactants. These nanoparticles are designed to encapsulate and protect bioactive compounds while enhancing their bioavailability and stability. SLNs exhibit several advantages for delivering bioactive compounds, including their small size, controlled release properties, and ability to protect sensitive compounds from degradation caused by environmental factors such as light, heat, or oxidation, enzymatic degradation, and instability caused by physiochemical factors.

The physicochemical properties of SLNs, including lipid composition, particle size, and surface charge, influence their stability and performance. A zeta potential greater than ±25 mV contributes to colloidal stability, minimizing aggregation and phase separation in liquid formulations. The structural integrity of SLNs, combined with precisely controlled formulation parameters, provides advantages over conventional liquid-based carriers, improving shelf stability and overall performance.

Thus, SLNs offer a promising solution to address challenges associated with the delivery of bioactive compounds, including vitamins, minerals, enzymes, algae-derived materials, proteins, peptides, amino acids, antioxidants, synthetic small molecules, and naturally derived phytochemicals such as botanical extracts and polyphenols. By encapsulating these compounds in a biocompatible lipid-based nanocarrier system, SLNs can protect sensitive bioactives from degradation due to oxidation, heat, enzymatic activity, or pH sensitivity, thereby enhancing bioavailability and enabling the controlled release of bioactive compounds compared to conventional formulations. The present invention utilizes a biocompatible lipid-based nanocarrier system that stabilizes bioactive compounds, protecting them from environmental degradation while optimizing solubility, sustained release, and targeted delivery.

SLNs offer a scalable and adaptable platform for pharmaceutical, nutraceutical, cosmetic, and food and beverage applications for living beings. Their ability to encapsulate both hydrophilic and lipophilic bioactives enables the development of customized formulations that enhance absorption, efficacy, and targeted delivery, making them a valuable system for improving bioactive compound delivery across multiple industries.

This invention enables the encapsulation of bioactives that are typically unstable in SLN delivery systems due to high molecular weight, oxidative sensitivity, or solubility limitations. These include algae-derived materials including red algae (Rhodophyta), brown algae (Phaeophyceae), green algae (Chlorophyta), any of their bioactive compounds, and any algae strain or bioactive compound that are newly identified or yet to be discovered algae strains and bioactive compounds, ensuring future adaptability and long-term innovation in SLN formulations. Algae-derived materials for encapsulation encompass cyanobacteria, such as Spirulina platensis and Aphanizomenon flos-aquae, which produce phycobiliproteins (C-phycocyanin, phycoerythrin), beta-carotene, gamma-linolenic acid (GLA), and polysaccharides. Green algae, such as Chlorella vulgaris and Scenedesmus obliquus, contain chlorophyll, lutein, zeaxanthin, polysaccharides, and antioxidant peptides. Red algae, such as Porphyridium cruentum, Gracilaria edulis, Chondrus crispus, and Kappaphycus alvarezii, provide sulfated polysaccharides, including carrageenan and agar, as well as bioactive lectins with immunomodulatory properties. Brown algae, such as Laminaria japonica, Ecklonia cava, and Sargassum fusiforme, contain fucoidan, phlorotannins, alginates, and other polyphenols known for their antioxidant and anti-inflammatory properties.

The invention further enables the encapsulation of marine microalgae, such as Isochrysis galbana and Phaeodactylum tricornutum, which contain fucoxanthin, docosahexaenoic acid (DHA), and eicosapentaenoic acid (EPA), bioactives with neuroprotective and cardiovascular health benefits. Additional microalgae, such as Tetraselmis chuii and Pavlova lutheri, provide lipids, proteins, sterols, and essential fatty acids that enhance systemic bioavailability and metabolic function. Thraustochytrids, including Schizochytrium sp. and Aurantiochytrium sp., produce DHA and omega-3 fatty acids, which are highly susceptible to oxidative degradation in conventional formulations. The present SLN system provides a protective lipid matrix that minimizes oxidation, extends systemic release, and enhances bioavailability of these compounds.

In addition to the above algae strains and bioactive compounds, the present invention further incorporates a broad spectrum of novel algae-derived compounds with significant potential for enhancing human health. These compounds, sourced from diverse microalgae and cyanobacteria strains, exhibit therapeutic, nutritional, and cosmetic properties, and may be encapsulated in SLNs to improve their bioavailability, stability, and targeted delivery.

Cyanobacteria, or blue-green algae, are particularly prolific producers of structurally diverse secondary metabolites. Among the notable compounds are cryptophycin 1, derived from Nostoc sp. GSV 224, a microtubule inhibitor with potent anticancer properties; and the tjipanazoles, indolocarbazole alkaloids from Tolypothrix tjipanasensis and Fischerella ambigua, which demonstrate protein kinase inhibition relevant to cancer and autoimmune therapies. Other promising bioactives include welwitindolinone A isonitrile from Hapalosiphon welwitschii, known for its neuroactivity and P-glycoprotein inhibition, and majusculamide C from Lyngbya majuscula, a cytotoxic compound with antifungal and anticancer potential. Additional compounds such as laxaphycin A and B from Anabaena, fischerellin A from Fischerella muscicola, calophycin from Calothrix fusca, and scytophycins from Scytonema pseudohofmanni display cytotoxic, antimicrobial, and anti-inflammatory activity, further supporting their utility in therapeutic and dermal applications.

From dinoflagellates, compounds such as amphidinol 2 from Amphidinium klebsi have shown strong antifungal properties, while yessotoxin (Dinophysis fortii) and goniodomin A (Alexandrium hiranoi) offer controlled therapeutic potential in oncology and immunology, when administered in precise doses. Okadaic acid and dinophysistoxin-1, though known as marine toxins, are powerful phosphatase inhibitors that may be repurposed under controlled delivery conditions to study or treat hyperproliferative diseases.

Bacillariophyta (diatoms) and Chrysophyta (golden algae) strains contribute polysaccharides and bioactives such as asterionellins from Asterionella sp., which may be useful in anti-aging, UV-protective, and antioxidant applications. Diatom-derived polysaccharides from Chaetoceros lauderi and Navicula delognei have also shown promise in immunomodulatory and wound-healing formulations.

Additionally, fatty acids such as r-linolenic acid from Dunaliella primolecta, Chloreococcum sp., and Stichococcus bacillaris are omega-3 compounds with known benefits in reducing inflammation, supporting cardiovascular health, and promoting skin barrier function. Compounds such as halogenated aromatics from Calothrix brevisima and cyanobacterin from Scytonema hofmanni further expand the range of bioactivities, including antibacterial and herbicidal effects, that may be safely utilized when encapsulated within SLNs.

The use of SLNs as a delivery platform provides multiple advantages for these bioactives. SLNs protect fragile compounds from oxidation and degradation, improve solubility, and allow for controlled and sustained release. This technology enables the encapsulation of hydrophobic and thermolabile compounds, facilitating oral, topical, transdermal, or systemic administration. The natural and sustainable origin of algae-derived compounds also aligns with increasing demand for clean-label, eco-friendly ingredients in both the pharmaceutical and nutraceutical sectors.

By integrating this expanded library of bioactive compounds and algae strains into SLN delivery systems, the present invention offers a novel and multifaceted approach to supporting multiple applications through natural, targeted, and sustainable interventions.

By incorporating structurally complex, highly reactive, and previously unstable algae bioactives, this SLN formulation expands the application of algae-derived compounds in pharmaceutical, nutraceutical, cosmetic, and functional food industries. Through advanced lipid stabilization and controlled-release technology, the present invention improves bioactive retention, reduces degradation, and ensures sustained bioavailability, addressing long-standing limitations of traditional delivery systems, making them ideal for pharmaceutical, nutraceutical, cosmetic, and food and beverage applications.

Encapsulation within SLNs helps shield bioactives from oxidation, enzymatic degradation, and environmental stressors, thereby prolonging shelf life and ensuring consistent delivery of bioactive compounds. The incorporation of bioactives into SLNs may also reduce the visibility of natural pigments in algae-derived compounds like Spirulina and phycocyanin, thereby increasing customer adoption. As an example, Spirulina-derived compounds present additional formulation challenges in food and nutraceutical applications due to their intense green pigmentation. Whole Spirulina biomass imparts a strong green hue, which can be undesirable in food products where neutral or lighter colors are preferred. Although there have been several products made out of Spirulina including energy bars, dips and spreads, cookies, and pasta, consumer adoption has been hindered by concerns over its green color. This color can negatively impact consumer perception and limit the commercial appeal of functional foods, beverages, and supplements. Many manufacturers seek to develop visually appealing, naturally colored products while retaining the nutritional benefits of Spirulina, highlighting the need for technologies that can mask or neutralize its color without compromising bioactivity. Spirulina, due to its nutrient content and anti-inflammatory properties can be utilized for ophthalmic eye drops for dry eyes offering a natural and sustainable alternative with synthetic steroids or less than effective therapies. However, due to Spirulina's intense green color, the applicability of this nutrient dense and therapeutic ingredient is currently not feasible for ophthalmic use without significant modifications. Additionally, Phycocyanin, a key bioactive protein-pigment found in Spirulina, is highly sensitive to degradation from light, heat, pH, and oxidation. This instability results in a loss of potency and structural integrity over time, limiting its use in pharmaceutical, biomedical, and dermatological applications. In therapeutic formulations, phycocyanin is susceptible to enzymatic degradation and oxidative stress, which can impact its efficacy as an anti-inflammatory, antioxidant, and immune-modulating agent. While its half-life varies depending on formulation and environmental factors, it tends to degrade more rapidly under elevated temperatures and acidic conditions. Additionally, as a large molecular-weight protein, its ability to penetrate the skin is limited, reducing its effectiveness in topical dermatological and cosmetic applications. Without effective stabilization and delivery strategies, maintaining phycocyanin's bioactivity and ensuring its efficient absorption for pharmaceutical and dermatological use remain significant challenges, necessitating advanced encapsulation and/or topical and transdermal delivery technologies. Additionally, phycocyanin has an intense blue color, which may limit consumer adoption in various applications such as therapeutics, cosmetics, and food and beverages. In our experiments, the creation of C-PC dispersions effectively removes the blue color, potentially expanding the applicability of C-PC across a wider range of therapeutic and commercial products.

SLNs provide a versatile delivery system, facilitating the administration of encapsulated bioactives through multiple routes of administration, including oral, transdermal, and topical, injectable, mucosal, ophthalmic, pulmonary, and suppository formulations. SLN-based formulations offer alternative delivery routes, including oral, transdermal, and inhalable applications, providing non-invasive administration options that improve patient compliance compared to traditional injections. Functionalized SLNs can be engineered with ligands or antibodies to enhance tissue-specific delivery, reducing immune suppression in healthy tissues. In pharmaceutical applications, SLNs can enhance drug stability, bioavailability, controlled release, targeted delivery, and patient compliance while minimizing toxicity, first-pass metabolism, and degradation. As an example, biologic immunomodulators, including TNF-alpha inhibitors, interleukin inhibitors such as guselkumab (IL-23 inhibitor), IL-6 inhibitors, and IL-12/23 inhibitors, B-cell inhibitors (Rituximab and Belimumab), T-cell inhibitors (Abatacept) and any other biologic not listed or discovered are widely used for treating autoimmune and inflammatory diseases such as psoriasis, rheumatoid arthritis, and inflammatory bowel disease (IBD). However, these biologics are available as intravenous (IV) infusion or subcutaneous (SC) injection due to the proteins' enzymatic degradation in the gut. IV formulations are invasive and pose challenges such as painful administration, infection risk, thrombophlebitis, and extravasation, while SC formulations are limited by small dosing volumes, slower absorption, injection site reactions, and variability in drug uptake, impacting patient compliance and therapeutic consistency. Encapsulating these immune modulators into SLNs can enhance stability, bioavailability, and targeted delivery while minimizing systemic side effects. The lipid matrix of SLNs protects biologics from enzymatic breakdown, extends drug half-life through sustained release, and enables precision targeting of inflamed tissues, reducing off-target immune suppression and improving therapeutic efficacy.

Certain modifications may be required for the successful encapsulation of biologics into SLNs due to their size, hydrophilicity, structural complexity, and sensitivity to processing conditions. Because biologics such as proteins, peptides, monoclonal antibodies (mAbs), and nucleic acids are generally large, hydrophilic, and structurally delicate, their incorporation into lipid-based nanoparticles necessitates specific formulation adjustments to maintain stability, bioactivity, and controlled release.

Examples of such modifications include hydrophilic core stabilization through lipid-polymer hybrid SLNs, surface functionalization to prevent aggregation or degradation, and cryoprotectant inclusion (e.g., trehalose, sucrose) to protect biologics during processing and storage. Cold homogenization, solvent evaporation, or microfluidization techniques may be used to minimize heat exposure, while mucoadhesive or pH-responsive coatings can enhance oral or transdermal absorption. Additionally, targeted ligand conjugation (e.g., FcRn-targeting for mAbs) can improve receptor-mediated uptake, and the selection of biocompatible lipids, surfactants, and stabilizers is crucial for optimizing encapsulation efficiency and preventing enzymatic degradation. These are examples and other modifications may exist or are yet to be discovered.

Additionally, SLNs can incorporate immune modulators into dissolvable microneedle (DMN) systems, allowing for painless, self-administrable topical or transdermal delivery with controlled release of bioactive compounds. By enhancing compound stability, controlling release, and enabling precision immune modulation, SLNs provide a drug delivery system for optimizing biologic therapies for autoimmune and inflammatory diseases.

In one embodiment, SLNs are embedded into advanced fabrics or wearable textiles to enable topical, intradermal, or transdermal delivery of bioactive compounds through prolonged skin contact. The SLNs described herein may be formulated using one or more lipids and surfactants, including natural, semi-synthetic, or synthetic materials, and may encapsulate a variety of active agents such as Spirulina-derived proteins, C-phycocyanin, vitamins, minerals, essential fatty acids, marine polyphenols, peptides, amino acids, antioxidants, or pharmaceutical compounds. Unlike traditional textile applications that release short-lived cosmetic actives or fragrances, the present invention enables controlled or sustained release of bioactives through garments such as socks, undergarments, gloves, wraps, or therapeutic fabrics. Depending on the formulation, active ingredients may act topically, intradermally or systemically. The combination of SLNs and fabric-based delivery systems offers a versatile platform for the application of bioactive compounds in a wide range of settings, such as personal care, medical, wellness, therapeutic, veterinary, performance, or industrial uses.

In certain embodiments, SLNs may encapsulate bioactive compounds derived from mushrooms, such as hericenones and erinacines from Hericium erinaceus (Lion's Mane), ergothioneine from Pleurotus ostreatus (oyster mushroom), and cordycepin from Cordyceps militaris. These bioactive compounds have been associated with neuroprotective, antioxidant, and metabolic regulatory activities. However, their natural bioavailability is limited due to poor solubility, degradation in the gastrointestinal tract, and low permeability across biological membranes. Encapsulation into SLNs may protect these compounds from premature degradation, improve their stability and solubility, enhance oral, transdermal, or mucosal permeability, and allow for controlled or sustained release.

In another example, psilocybin, a prodrug of psilocin with therapeutic potential for psychiatric and neurological disorders, faces challenges related to low bioavailability, rapid metabolism, and first-pass degradation. Encapsulation of psilocybin into SLNs enhances its stability, absorption, and controlled release, providing a sustained therapeutic effect while reducing gastrointestinal side effects. SLN formulations protect psilocybin from enzymatic breakdown, enabling higher systemic bioavailability at lower doses and improved brain targeting through lipid-based transport mechanisms. Additionally, SLNs facilitate alternative delivery routes of psilocybin, including transdermal delivery systems, intranasal sprays, and sublingual films, offering non-invasive, controlled-release psychedelic-assisted therapy for conditions such as depression, PTSD, and neurodegenerative diseases.

This invention relates to the nanoencapsulation of neuroactive compounds within solid lipid nanoparticles (SLNs) to enhance stability, bioavailability, and controlled release, thereby improving therapeutic applications. The disclosed SLN-based formulations address significant challenges associated with various psychoactive, dissociative, cannabinoid, and neuroactive agents, including rapid metabolism, poor solubility, chemical instability, degradation under physiological or storage conditions, and limited administration routes. While SLNs have been traditionally used for lipophilic drugs, this invention expands their utility through modifications to lipid composition, surfactants, polymer-lipid hybridization, and co-encapsulation strategies, allowing for the effective nanoencapsulation of both lipophilic and hydrophilic neuroactive drugs.

The neuroactive compounds suitable for SLN encapsulation include psychedelic tryptamines such as DMT (N,N-Dimethyltryptamine) and 5-MeO-DMT, kappa-opioid receptor agonists such as Salvinorin A and any agents that have yet to be discovered. These compounds hold therapeutic potential in addressing mental health disorders, chronic pain, neuroinflammation, and neuroplasticity-related conditions, but they are often limited by low oral bioavailability, instability, and short duration of action. The disclosed SLN formulations provide a biocompatible and biodegradable lipid matrix that enhances drug protection from oxidation, enzymatic degradation, and first-pass metabolism, while also enabling sustained or controlled release to optimize therapeutic effects.

SLN-based nanoencapsulation provides several advantages in improving the pharmacokinetics and administration of these neuroactive agents. DMT and 5-MeO-DMT, which are rapidly metabolized by monoamine oxidase (MAO) and exhibit poor oral bioavailability, can be optimized for SLN encapsulation through co-encapsulation with MAO inhibitors (e.g., harmine, harmaline), ion pairing with lipid carriers, or phospholipid modifications to improve transdermal and mucosal absorption. Salvinorin A, a highly lipophilic kappa-opioid agonist with an ultra-short half-life, can benefit from SLNs with mucoadhesive coatings or polymeric lipid matrices to prolong systemic circulation and improve transmucosal absorption through intranasal or sublingual administration. This invention also contemplates customizing SLN compositions to expand their applicability to a wider range of neuroactive compounds, including hydrophilic drugs or those with complex pharmacokinetics. SLN modifications may include hybrid lipid-polymer nanoparticles to accommodate both hydrophilic and lipophilic active compounds, co-encapsulation with bioenhancers (e.g., terpenes, surfactants, or permeation enhancers) to improve transdermal and mucosal absorption, hydrophobic ion pairing techniques to enhance encapsulation of charged molecules and peptides, and functionalized lipid carriers that allow for targeted CNS delivery, including strategies to enhance blood-brain barrier permeability. Additionally, this invention is not limited to the psychedelics and dissociatives but may be applied to other central nervous system (CNS)-acting drugs, neuropeptides, neurotransmitter enzymes, psychoplastogens, and emerging psychoactive therapies that require enhanced stability, bioavailability, and targeted delivery.

By leveraging solid lipid nanoparticle-based drug delivery, this invention provides a scalable, biocompatible, and adaptable platform for neuroactive drug administration, enabling oral, transdermal, intranasal, sublingual, and injectable formulations with enhanced therapeutic efficacy. The disclosed SLN formulations have broad applications in psychedelic-assisted therapy, chronic pain management, neurodegenerative disease treatment, and personalized medicine, offering a next-generation drug delivery system that improves patient compliance, therapeutic precision, and clinical applicability.

In the nutraceutical space, SLNs can enhance the absorption and controlled release of poorly bioavailable compounds, such as non-heme iron and vitamin B12, by improving solubility and potentially bypassing first-pass metabolism. For individuals with conditions like pernicious anemia or iron-deficiency anemia, SLN-based formulations may offer an alternative to invasive injections by providing sustained, controlled release of these essential nutrients. vitamin K plays a vital role in blood clotting, bone metabolism, and cardiovascular health, yet its bioavailability and stability are significantly limited by poor solubility, rapid metabolism, and susceptibility to oxidation. Encapsulating vitamin K1 (phylloquinone) and K2 (menaquinone) into SLNs enhances its absorption, stability, and controlled release, overcoming the challenges of short systemic circulation time and degradation under light, heat, and oxygen exposure. The lipid matrix of SLNs improves intestinal uptake, extends systemic retention, and allows for organ-specific targeting, such as bone tissue for osteoporosis prevention or vascular tissues for reducing arterial calcification.

SLN encapsulation of vitamin K provides controlled and sustained release, ensuring consistent therapeutic levels while reducing dosing frequency. This technology enhances oral bioavailability for dietary supplementation, transdermal absorption for non-invasive delivery, and intravenous formulations for clinical applications. The protective lipid barrier also prevents oxidation and degradation, significantly improving shelf life and formulation stability. By improving the bioavailability, stability, and therapeutic efficacy of vitamin K, SLN-based delivery systems offer a superior alternative to conventional supplementation and pharmaceutical formulations.

Likewise, magnesium plays a crucial role in over 300 enzymatic reactions in the body, affecting muscle function, nerve signaling, cardiovascular health, metabolism, and bone density. However, its bioavailability, absorption, and stability vary significantly depending on the magnesium form used. Encapsulating magnesium into SLNs can overcome common challenges, such as low solubility, rapid elimination, gastrointestinal irritation, and poor cellular uptake.

Chromium, which plays a critical role in glucose metabolism, has low bioavailability in conventional forms and is often ineffective in dietary supplements. Finally, manganese, crucial for connective tissue formation and bone health, faces similar absorption issues, making it difficult to deliver effectively in typical formulations. These bioactive compounds face challenges related to solubility, absorption, and stability that hinder their therapeutic potential and effectiveness in conventional products.

In certain embodiments, the bioactive compound embedded in the dissolvable microneedle system may comprise one or more plant-derived actives. These include botanical extracts, essential oils, and plant-derived volatile compounds. Botanical extracts may be obtained from leaves, roots, stems, flowers, or fruits of medicinal or aromatic plants, and may include water-soluble or alcohol-soluble phytochemicals such as flavonoids, alkaloids, tannins, glycosides, saponins, and polyphenols. Essential oils and related volatile compounds may include whole oils or individual constituents such as terpenes (e.g., linalool, limonene), aldehydes (e.g., cinnamaldehyde), esters (e.g., linalyl acetate), ketones (e.g., carvone), and oxides (e.g., 1,8-cineole). These compounds may be included for their therapeutic, cosmetic, or aromatic properties, including anti-inflammatory, antimicrobial, analgesic, anxiolytic, antioxidant, or fragrance-enhancing effects. Essential oils or their constituents may optionally be encapsulated in delivery vehicles such as solid lipid nanoparticles (SLNs), nanostructured lipid carriers (NLCs), polymeric nanoparticles or any other nanocarrier prior to incorporation into the microneedle matrix to enhance stability, solubility, and controlled release. The invention contemplates the use of such plant-derived volatile compounds in transdermal, dermal, mucosal, or scalp applications, either alone or in combination with other bioactive ingredients.

In dermatological and cosmetic applications, SLNs can facilitate the topical delivery of large-molecule bioactives, such as peptides and proteins, by enhancing skin penetration and stability, thereby offering antioxidant, anti-aging, and anti-inflammatory benefits.

Algae-based materials, including various algae strains and their bioactive compounds, hold significant potential for applications in dermatology and cosmetics due to their rich antioxidant, anti-inflammatory, and skin-rejuvenating properties. However, their widespread use has been limited by challenges such as poor stability under environmental conditions (light, heat, and oxidation), low skin permeability due to hydrophilic nature, and formulation incompatibilities related to pH sensitivity, odor, and pigmentation issues. Overcoming these limitations through advanced delivery systems, such as solid lipid nanoparticles (SLNs), can enhance the stability, bioavailability, and efficacy of algae-derived bioactives, unlocking their full potential in skincare and dermatologic applications. Phycocyanin, a water-soluble pigment derived from Spirulina, possesses potent antioxidant, anti-inflammatory, and skin-brightening properties, making it a promising active ingredient for cosmetic and dermatologic formulations. However, its direct application in skincare products is significantly limited by several factors, including instability under environmental conditions, poor skin penetration, short shelf-life, and pH incompatibility with many formulations. Phycocyanin is highly susceptible to degradation when exposed to light, heat, and oxygen, leading to rapid loss of efficacy and color fading. Additionally, due to its hydrophilic nature, phycocyanin demonstrates poor transdermal absorption, limiting its bioavailability in deeper skin layers where it can exert its therapeutic effects. Moreover, its strong blue pigmentation presents potential formulation challenges, such as staining the skin or fabric, further restricting its commercial application.

To overcome these challenges, the encapsulation of phycocyanin into SLNs presents a highly effective delivery system that enhances its stability, bioavailability, and controlled release. SLNs provide a protective lipid matrix that shields phycocyanin from degradation due to environmental factors, thereby significantly extending its shelf life. The lipid composition of SLNs closely mimics the skin's natural structure, facilitating enhanced penetration into the epidermal and dermal layers, improving its therapeutic potential. Furthermore, SLNs enable controlled and sustained release of phycocyanin, ensuring prolonged antioxidant and anti-inflammatory activity, reducing the need for frequent application. The encapsulation process also stabilizes phycocyanin across a wider range of pH conditions, enhancing its compatibility with various cosmetic formulations, including creams, serums, lotions, and emulsions.

Spirulina, a microalga rich in bioactive compounds, has significant potential in dermatologic and cosmetic formulations due to its antioxidant, anti-inflammatory, and skin-nourishing properties. It contains essential nutrients such as phycocyanin, carotenoids, polysaccharides, all 9 essential amino acids, vitamins, and minerals, which contribute to skin hydration, protection against oxidative stress, and enhanced skin elasticity. However, direct incorporation of Spirulina into skincare products presents several challenges, including instability under environmental conditions, poor skin penetration, strong odor, and undesirable green-blue pigment. Spirulina's bioactive components are susceptible to degradation when exposed to light, heat, and oxygen, leading to reduced efficacy and shelf life. Additionally, its hydrophilic nature limits transdermal absorption, restricting its ability to exert benefits beyond the skin surface. The characteristic blue-green pigmentation and marine odor further complicate formulation, potentially affecting consumer acceptance.

The encapsulation of Spirulina into SLNs provides an effective strategy to overcome these challenges and enhance its cosmetic application. SLNs provide a protective lipid matrix that stabilizes Spirulina's bioactives, shielding them from environmental degradation and significantly extending their shelf life. The lipid composition of SLNs mimics the skin's natural barrier, facilitating improved penetration of Spirulina's beneficial compounds into deeper skin layers, thereby enhancing bioavailability and efficacy. Furthermore, SLNs enable controlled and sustained release of Spirulina-derived antioxidants, peptides, amino acids, vitamins, and minerals ensuring prolonged skin benefits while reducing the frequency of application. This encapsulation technique also mitigates the challenges associated with Spirulina's odor and pigmentation by the lipid, which will appear neutral (yellow/off-white) during application improving visual and sensory appeal.

The lipid matrix used in SLN formulations not only enhances stability but also provides additional skin-conditioning benefits. SLNs offer a smooth, lightweight texture that blends seamlessly into creams, serums, masks, and emulsions without leaving a greasy residue. The lipid carrier system interacts synergistically with other bioactive compounds, such as ceramides, vitamin E, and natural oils, to further support skin hydration and barrier function. Additionally, encapsulated Spirulina is more compatible with a wider range of pH conditions, allowing its incorporation into diverse skincare formulations while maintaining bioactivity. Given these advantages, nanoencapsulated Spirulina presents a scientifically advanced and consumer-friendly solution for addressing oxidative stress, skin aging, hydration deficiencies, environmental damage, and accelerated post-surgical wound care for the face and other parts of the body. Its incorporation into dermatologic and cosmetic applications represents a natural and sustainable approach to enhancing skin health and beauty through advanced nanoencapsulation.

Ecklonia cava, a marine-derived brown algae rich in phlorotannins, polyphenols, polysaccharides, and essential minerals, has demonstrated significant potential for hair and scalp health due to its antioxidant, anti-inflammatory, and hair growth-promoting properties. However, its widespread application in hair care formulations has been limited by challenges such as poor stability under environmental conditions, low bioavailability, and limited scalp penetration. Nanoencapsulation of Ecklonia cava extracts into solid lipid nanoparticles (SLNs) addresses these challenges, enhancing stability, controlled release, and transdermal absorption, thereby maximizing the efficacy of Ecklonia cava in hair care applications.

SLN-encapsulated Ecklonia cava provides superior antioxidant protection, neutralizing oxidative stress that contributes to hair follicle aging and hair loss. Its phlorotannins and polyphenols shield hair follicles from environmental aggressors such as UV radiation, pollution, and free radicals, preserving scalp health and hair vitality. Additionally, SLNs improve bioavailability and controlled release, ensuring that Ecklonia cava's bioactives are delivered gradually over time, prolonging their effectiveness in stimulating hair growth, reducing inflammation, and maintaining scalp hydration.

Ecklonia cava has also been shown to inhibit the enzyme 5-alpha reductase, which is responsible for the conversion of testosterone to dihydrotestosterone (DHT), a key factor in androgenetic alopecia (hair thinning and loss). Encapsulation into SLNs enhances targeted delivery to hair follicles, increasing the efficacy of DHT inhibition, thereby supporting stronger, thicker, and healthier hair growth. Moreover, Ecklonia cava's anti-inflammatory properties help soothe the scalp, reduce itching, and alleviate conditions such as dandruff, seborrheic dermatitis, and scalp irritation.

SLN-based encapsulation further enhances Ecklonia cava's hydration and scalp-conditioning effects, as its polysaccharides and essential minerals help retain moisture, strengthen the hair shaft, and reduce breakage and brittleness. The lipid matrix of SLNs also mimics the scalp's natural oils, ensuring better penetration and sustained nourishment of hair follicles. Additionally, nanoencapsulation prevents Ecklonia cava's bioactives from oxidizing or degrading, significantly extending the shelf life and effectiveness of hair care formulations.

By leveraging SLN technology, Ecklonia cava can be effectively incorporated into shampoos, conditioners, scalp serums, and hair masks, offering advanced hair rejuvenation, anti-aging, and protective benefits. This innovative approach allows for the optimized delivery of Ecklonia cava bioactives, promoting scalp health, reducing hair loss, and enhancing overall hair quality, making it a high-performance ingredient in modern hair care formulations.

In food and beverage applications, SLNs offer a solution to stabilize Spirulina-derived compounds, improving their taste, bioavailability, and shelf life. SLNs also allow for the incorporation of Spirulina or phycocyanin into non-green functional foods like pasta, cookies, and energy drinks, without the intense green color that typically comes with Spirulina. SLN-encapsulated Spirulina or phycocyanin can enhance plant-based proteins in protein bars and meat alternatives, providing additional nutritional benefits. SLNs also stabilize water-soluble vitamins and antioxidants in functional beverages, ensuring enhanced bioavailability and stability.

Finally, SLNs provide specialized nutrient formulations for individuals with conditions such as HIV, anorexia, and gastrointestinal disorders, which may require enhanced protein and micronutrient delivery. SLNs can be utilized for targeted delivery of bioactive compounds, providing higher bioavailability, prolonged stability, and sustained therapeutic action across various industries.

SLNs may be used with a dissolvable microneedle system (described below) to enhance topical and transdermal delivery and improve absorption.

SLNs can be formulated for a wide range of delivery methods, including oral (capsules, powders, functional beverages, edible films), transdermal (patches, infused textiles like socks), topical (creams, serums, wound healing gels, hair care products), injectable (IV, IM, SC biologic-loaded nanoparticles, hydrogel implants), mucosal (eye drops, nasal sprays, buccal tablets, inhalable powders, transbuccal films), and suppositories (rectal and vaginal formulations), and ocular formulations. These diverse delivery systems enhance bioavailability, stability, and controlled release, making SLNs a valuable platform technology across the pharmaceutical, nutraceutical, cosmetic, and food and beverage, veterinary, and agricultural industries for humans and animals.

One embodiment involves incorporating SLNs into dissolvable microneedle (DMN) systems, enabling painless, transdermal nutrient delivery while bypassing the gastrointestinal tract. This delivery system could be particularly beneficial for individuals with malabsorption disorders such as Celiac disease, Crohn's disease, and cystic fibrosis, elderly populations, and patients requiring non-oral supplementation as well as those with autoimmune disorders, and micronutrient deficiencies. It also increases patient compliance with medication regimens.

The present invention introduces optimized SLN formulations designed for broad bioactive encapsulation, enhanced transdermal and systemic absorption, and integration into advanced delivery systems. By offering both standalone SLN nanoencapsulated ingredients and DMN-integrated applications, this technology provides a scalable and versatile solution for the pharmaceutical, nutraceutical, cosmetic, food and beverage, veterinary, and agricultural industries. The disclosed innovation overcomes key limitations of conventional delivery systems by enhancing the stability, bioavailability, and targeted delivery of bioactive compounds. This advancement enables more effective and versatile applications across pharmaceuticals, nutraceuticals, cosmetics, food and beverage, and transdermal delivery systems, offering a transformative solution for improved product efficacy and consumer accessibility.

### Parameters, Materials, and Structure of a SLN

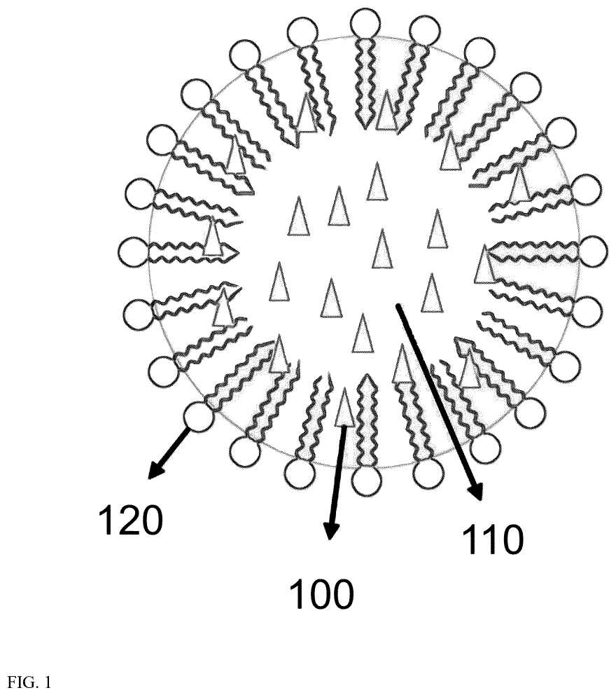

FIG. 1 shows a diagram of a SLN. As shown, the bioactive component 100 is distributed within a lipid matrix 110, which is stabilized by a surfactant layer 120. In an embodiment, the SLN's have a mean particle size of less than 350 nm, a polydispersity index (PDI) of less than 0.30, and an absolute value of zeta potential of greater than 25 mV.

The particle size of the SLN is less than 1000 nm. In an embodiment, the size can be less than 350 nm. Nanovesicles with a diameter of 350 nm or below can deliver their contents to some extent into the deeper skin layers when delivered topically. SLNs of this diameter can act like a storage “depot” of bioactive ingredients for better sustained release. In an embodiment, the particle size may be 200 nm or less. Nanoparticles with a particle size of 120 nm or less are able to cross the skin and enter into systemic circulation.

SLNs with a size range of 500 to 1000 nm can be utilized for specific topical and oral applications, particularly where controlled release, surface protection, and enhanced bioactive stability are desired. In topical formulations, larger SLNs remain on the epidermal surface rather than penetrating the deeper layers of the skin, making them particularly effective for moisturizing creams, sunscreens, occlusive barrier treatments, and protective skincare formulations. Their larger size allows for sustained release of bioactives, providing long-lasting hydration and antioxidant effects while minimizing transepidermal water loss. However, SLNs in the 500 nm-1000 nm range are less suitable for transdermal drug delivery or deep-penetrating cosmeceuticals, as their absorption into the lower dermal layers is limited.

For oral applications, 500 to 1000 nm SLNs can encapsulate sensitive bioactives, enhancing their stability and controlled release within the gastrointestinal tract. These larger nanoparticles are beneficial for delayed-release formulations, gut-targeted nutraceuticals, and protection of active compounds from gastric degradation. However, their absorption into the systemic circulation may be lower compared to smaller nanoparticles (<200 nm), which can more effectively cross intestinal barriers. Despite this, SLNs in this size range offer advantages for probiotics, lipid-soluble vitamins, and polyphenolic compounds by improving bioavailability and ensuring prolonged therapeutic effects.

Polydispersity Index (PDI) is a parameter used to measure the width of the particle distribution in a solution and is a measure of the homogeneity of particle size within the dispersed system. A PDI value of 0 indicates that all particles are of the same size, while a value of 1 indicates that there is a wide range of particle sizes in the sample (i.e., the smaller the PDI, the more uniform the system). A PDI of less than 0.30 ensures a relatively uniform particle size distribution, reducing aggregation risks and promoting effective delivery.

Zeta potential (ZP) is used to describe the stability of a dispersion system. In general, the greater the absolute value of the potential, the more stable the dispersion system. This stability is because the dispersion system has more like charges, leading to higher electrostatic repulsion between molecules, which reduces the likelihood of particle aggregation. The system is deemed stable when the absolute value of zeta potential is greater than 25 mV, indicating that the nanoparticles have sufficient charge to repel one another, thus preventing aggregation and promoting transdermal diffusion. Providing a nanoparticle with a zeta potential less than-25 mV not only prevents agglomeration and promotes stability but also enhances transdermal diffusion by minimizing interactions between negatively charged nanoparticles and negatively charged endothelial cells in the skin.

Encapsulation efficiency (EE %) is a measure of how effectively an active compound is incorporated into a delivery system, such as nanoparticles, such as an SLN, relative to the total amount initially introduced. It reflects the formulation's ability to retain the active ingredient within the carrier while minimizing its loss in the external phase. Encapsulation efficiency is determined by the ratio of the encapsulated compound to the total compound used, expressed as a percentage. A higher encapsulation efficiency signifies better incorporation and retention of the active compound, which is essential for improving stability, bioavailability, and controlled release in pharmaceutical, nutraceutical, and cosmetic applications.

Mean SLN particle size, PDI, zeta potential, and EE are compared across all SLN constructs based upon a) target particle size and b) the lipid and surfactant used to make the SLNs. Least square means and difference of least square means, standard errors, a 95% confidence interval for the mean difference, and p-value are determined. All data is analyzed in log units. All statistical testing is two-sided with a significance level of 5%.

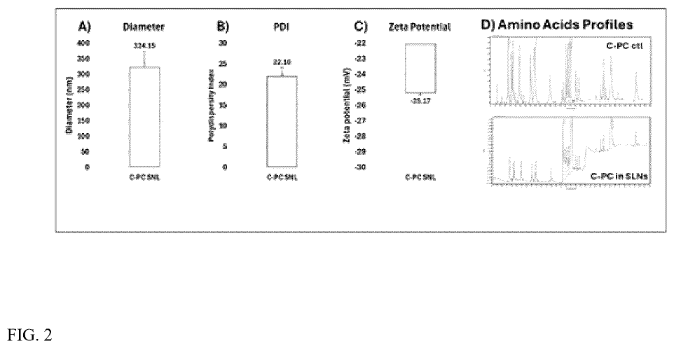

FIG. 2 is a graph showing mean SLN particle size, PDI, zeta potential, and EE for C-PC based SLNs manufactured according to the processes described below.

The lipids comprising the lipid matrix 110 can be natural or semi-synthetic lipids such as long-chain saturated fatty acids (e.g., stearic acid, behenic acid, palmitic acid), triglycerides and monoacylglycerols (e.g., tripalmitin, tristearin, glyceryl monostearate), and natural waxes (e.g., cocoa butter, shea butter, beeswax, carnauba wax), selected to enhance stability, EE, bioavailability, and controlled release of encapsulated bioactive compounds in SLN formulations. Other lipids that may be used include fatty alcohols such as cetyl alcohol; triglycerides such as tri-stearin; partial glycerides such as Imwitor; steroids such as cholesterol; and waxes such as candelilla wax and cetyl palmitate. Synthetic lipids that may be used are PEGylated lipids (PEGylated phospholipids, glyceryl behenate, trimyristin), glyceryl dibehenate, glyceryl distearate, and cetyl palmitate. It is to be understood that the above-listed materials are not an exclusive list and that other lipids may also be used, as long as they remain solid at body temperature.

In an embodiment, to avoid the possibility that some ingredients may potentially cause allergic reactions in sensitive subjects, the SLN may comprise only 100% naturally occurring lipids, such as stearic acid, palmitic acid or cholesterol.

In an embodiment, two or more lipids may be used in the fabrication of SLNs. A mixture of two or more lipids may be used in the fabrication of SLNs to enhance stability, drug loading efficiency, controlled release properties, and biocompatibility. Lipid combinations may include glyceryl monostearate and stearic acid to improve crystallinity and prevent polymorphic transitions, glyceryl behenate and phosphatidylcholine to enhance stability and bioavailability, or Precirol ATO 5 and medium-chain triglycerides (MCTs) to optimize drug solubility and controlled release. Other examples include blends of tristearin and lecithin, cetyl palmitate and phospholipids, or stearic acid and oleic acid, which can be selected based on their physicochemical properties to achieve desired nanoparticle characteristics. Any combination of lipids may be used to enhance the stability, drug loading efficiency, controlled release properties, and biocompatibility of the SLN.

The bioactive component 100 can be any bioactive compound that can be dissolved in water or another solvent, and that can be nanosized if its particle size is 350 nm or above. The bioactive compound 100 could be a vitamin, mineral, enzyme, algae-derived material, protein, peptide, amino acid, antioxidant, synthetic small molecule, or a naturally derived phytochemical compound, including botanical extracts and polyphenols. Preferentially, the bioactive component 100 is a compound with poor oral bioavailability, one that undergoes rapid degradation, or one that requires enhanced stability, controlled release, targeted delivery, or improved cellular uptake.

This invention also relates to the co-encapsulation of multiple bioactive compounds within SLNs to enhance stability, bioavailability, and controlled or sustained release. The disclosed SLN formulations address common challenges in drug delivery, including rapid metabolism, poor solubility, systemic toxicity, and inconsistent therapeutic effects. By enabling the simultaneous delivery of complementary or synergistic agents, these formulations optimize therapeutic efficacy while reducing adverse effects. This technology applies to a wide range of bioactive compounds, such as those targeting pain management, metabolic regulation, hormonal balance, neuroprotection, and immune support. While specific formulations are described herein, the invention may include other combinations that have been discovered or are yet to be discovered, allowing for broad adaptability across various therapeutic applications.

SLNs provide an ideal lipid matrix for encapsulating lipophilic compounds, while hydrophilic compounds require formulation modifications, such as lipid-polymer hybridization, surfactant-assisted encapsulation, or hydrophobic ion pairing, to achieve optimal encapsulation efficiency. Various SLN formulation techniques, including nano-chelation, polymeric coatings, and controlled-release modifications, ensure compound stability, sustained therapeutic action, and targeted drug delivery. The following examples illustrate highly feasible, novel SLN formulations that provide enhanced clinical benefits while demonstrating non-obvious and synergistic combinations of bioactive agents.

A ketamine+lidocaine SLN formulation is designed for transdermal or injectable pain relief, providing dual-phase analgesia through the immediate action of lidocaine and sustained-release ketamine, reducing systemic toxicity while enhancing localized pain relief. Since both ketamine and lidocaine are lipophilic, SLNs allow for their gradual absorption and prolonged analgesic effects, making this formulation ideal for neuropathic pain, musculoskeletal pain, and post-surgical recovery. Another novel example is a melatonin+apigenin+magnesium SLN, which offers gradual sleep induction and cortisol regulation, leveraging sustained melatonin release, cortisol-lowering apigenin, and nano-chelated magnesium to enhance neuromodulation and support relaxation. While melatonin and apigenin are lipophilic and easily encapsulated in SLNs, magnesium requires nano-chelation or polymeric lipid stabilization to ensure controlled release and absorption.

For hormonal balance and metabolic health, a DHEA+phosphatidylserine+berberine SLN combines a hormone precursor, a cortisol regulator, and an insulin sensitizer to support hormone balance, stress reduction, and metabolic function. DHEA and phosphatidylserine, both lipid-soluble, are naturally suited for SLNs, while berberine, a moderately water-soluble compound, requires surfactant-assisted encapsulation to enhance its bioavailability. Another innovative formulation, a berberine+capsaicin+EGCG SLN, provides a sustained-release thermogenic and insulin-regulating system, optimizing fat metabolism, blood sugar control, and energy expenditure while preventing the gastrointestinal irritation commonly associated with berberine and capsaicin. SLNs help stabilize EGCG (green tea polyphenol), improve berberine absorption, and protect capsaicin from premature degradation, ensuring a gradual, sustained thermogenic effect.

The invention encompasses any co-encapsulation of multiple bioactive compounds within SLNs, including those developed for neurological health, immune modulation, gut microbiome support, and advanced drug delivery systems. While certain compounds may naturally align with lipid-based encapsulation, others requiring polymer-lipid hybridization, nano-chelation, or emulsifier-assisted stabilization are also within the scope of this technology. This ensures that hydrophilic, lipophilic, and amphiphilic compounds can be co-formulated into a single, stable SLN system for multi-functional drug delivery.

The claims outlined in this invention include the co-encapsulation of multiple bioactive compounds within a solid lipid nanoparticle system, enabling optimized pharmacokinetics, enhanced bioavailability, and targeted release. This may include SLNs formulated with ketamine and lidocaine for dual-phase pain relief, melatonin and apigenin for sleep optimization, DHEA and phosphatidylserine for hormonal regulation, and berberine and capsaicin for metabolic enhancement. However, the invention is not limited to these specific formulations and encompasses other bioactive combinations that address a variety of health conditions. By leveraging SLN technology, this invention provides a scalable and adaptable platform for the efficient delivery of multiple active agents, offering advanced therapeutic solutions in pharmaceuticals, nutraceuticals, and personalized medicine.

In an embodiment, the bioactive component 100 comprises bioactive compounds derived from Spirulina. Spirulina (Arthrospira platensis) is a free-floating, filamentous cyanobacterium that is widely used as a dietary supplement and functional food ingredient due to its high nutritional content and therapeutic properties. It is rich in proteins, essential amino acids, polyunsaturated fatty acids, vitamins, and antioxidants, making it a promising bioactive source for pharmaceutical, nutraceutical, and cosmetic applications. Active components of Spirulina, such as C-Phycocyanin (C-PC), is a potent antioxidant that has anti-inflammatory and immunomodulatory properties. C-PC has been shown to inhibit oxidative stress, suppress inflammatory pathways (such as COX-2 and NF-kB), and promote cellular protection, making it a valuable agent for anti-aging skin care, wound healing, and inflammatory conditions. However, C-PC is sensitive to light, heat, enzymatic degradation, and oxidative stress, limiting its effectiveness in formulations. Due to the instability of C-PC, it is desirable to encapsulate it into a SLN. Encapsulation within an SLN protects C-PC from degradation, enhances its bioavailability, and enables controlled release prolonging its activity and improving transdermal penetration in topical applications. SLN fabrication also masks its blue pigment which makes it desirable for topical and food & beverage applications.

Spirulina is a complex mixture of bioactives, including hydrophilic (e.g., phycocyanin) and lipophilic components, making Spirulina more challenging than other materials (e.g., beta-carotene) to encapsulate in solid lipid nanoparticles (SLNs). For example, encapsulation of beta-carotene primarily focuses on oxidation prevention and achieving uniformity, often resulting in smaller particle sizes and lower polydispersity index (PDI) due to its simpler composition. In contrast, Spirulina requires dual-phase stabilization strategies to accommodate its diverse compounds, leading to more complex processing methods and a need for customized stabilizers to ensure particle uniformity and stability. Additionally, Spirulina bioactives have higher susceptibility to degradation under environmental stress (light, pH, and temperature), requiring stabilization approaches that are unnecessary for single-compound formulations like beta-carotene SLNs.

Spirulina (Arthrospira platensis), a nutrient-dense cyanobacterium, has been extensively studied for its therapeutic, nutritional, cosmetic, and agricultural applications. Due to its rich composition of proteins, vitamins, minerals, antioxidants, and bioactive compounds, Spirulina has been investigated for its potential health benefits in cardiovascular health, immune modulation, neuroprotection, metabolic regulation, and inflammation management.

In cardiovascular health, clinical studies have shown that Spirulina supplementation can improve lipid profiles, leading to reductions in low density lipoprotein (LDL) cholesterol, triglycerides, and blood pressure, which may contribute to reduced cardiovascular disease risk. Additionally, systematic reviews and meta-analyses suggest its potential role in weight management, demonstrating significant reductions in body weight, body mass index (BMI), and waist circumference. In the area of immune system modulation, Spirulina has been investigated for its potential as an adjunct therapy in HIV-1 management, where high-dose supplementation has been associated with reduced viral load and increased CD4 cell counts.

Spirulina has also demonstrated neuroprotective properties, particularly in cognitive function, as evidenced by clinical trials showing improvements in memory, visual learning, and working memory in individuals with mild cognitive impairment following daily supplementation. Its anti-inflammatory and antioxidant properties have been evaluated in inflammatory conditions such as ulcerative colitis, where supplementation resulted in reduced disease activity and improved quality of life. Additionally, Spirulina has been explored for respiratory health, with evidence suggesting beneficial effects on allergic rhinitis symptoms due to its immunomodulatory activity. Spirulina has also been studied for its antiviral properties, with emerging research indicating potential effects against viral infections through its ability to modulate immune responses.

Additionally, clinical trials also support the use of Spirulina as an anti-inflammatory agent. In a clinical trial for chronic periodontitis, Spirulina gel applied with the Scaling and Root Planing (SRP) treatment significantly improved the clinical outcome, likely associated with the anti-inflammatory activity of C-PC in Spirulina. Another clinical trial in patients with oral submucous fibrosis (OSMF) reported that daily oral administration of Spirulina significantly reduced the mouth stiffness, oral ulcers, and erosions associated with the disease. Spirulina also showed a promising functionality as adjuvant chemotherapy to improve the immune function of tumor patients by significantly increasing IgM levels and CD8+ T cell numbers As C-PC is one of the major bioactive ingredients in Spirulina, these clinical data support its therapeutic potential as an anti-inflammatory natural compound. Supported by the promising clinical outcomes, there is an ongoing clinical trial evaluating the efficacy of C-PC against the neurotoxicity of oxaliplatin-based chemotherapy in patients with gastrointestinal cancer.

In addition to its therapeutic applications, Spirulina has been extensively studied for its potential to improve nutrition and development in children, particularly in developing countries. Clinical trials in Zambia, Cambodia, and India have examined Spirulina's impact on growth, anemia, and immune function in malnourished children. A study in Zambia found that infants receiving Spirulina supplementation were less likely to develop a cough and showed improved motor development milestones. In Cambodia, Spirulina supplementation was associated with a modest trend toward higher weight gain and a reduction in anemia prevalence. In India, intervention studies showed that Spirulina supplementation significantly reduced severe wasting and stunting in young children while improving mid-upper arm circumference measurements, indicating enhanced nutritional status. While the effects on physical growth metrics such as height and weight gain have been variable, these studies support Spirulina's potential role in addressing malnutrition and immune health in vulnerable populations.

Beyond its medical applications, Spirulina has commercial applications in the cosmetic industry, where it serves as a natural colorant in personal care products due to its high content of phycocyanin and carotenoids. In agriculture and aquaculture, Spirulina is widely used as an animal feed supplement, enhancing the nutritional value and growth rates of livestock, poultry, and aquatic species.

Given its wide-ranging applications and bioactive properties, Spirulina is an important bioactive compound for SLNs fabrication. Encapsulation can improve bioavailability, stability, and controlled release, allowing for enhanced therapeutic and cosmetic applications while addressing stability and degradation concerns.

C-Phycocyanin (C-PC) is a potent anti-inflammatory natural compound by itself. Phycocyanin is an FDA-approved water-soluble, non-toxic, and blue-colored photosynthetic pigment, which has undergone rigorous safety evaluations. In various color additive petitions, FDA has determined an Acceptable Daily Intake (ADI) of 1.0-1.8 grams/person/day (g/p/d), for phycocyanin, and a No Observed Effect Level (NOEL) of 108-184.5 g/p/d in a GRAS submission for phycocyanin. It has been used in food, cosmetics, and pharmaceutical industries. C-PC, a major active compound in Spirulina, has also been widely used as a nutritional supplement. The anti-inflammatory function of C-PC has been extensively investigated. For example, oral administration of C-PC significantly reduced intestine inflammation and colitis in animal models. C-PC also showed promising efficacy in modulating organ inflammation beyond the digestive system. In animal models, C-PC, delivered via nasal spray or oral administration, showed the potential to reduce inflammation in diseases of the liver, lungs, and brain. Although the molecular mechanism of C-PC's anti-inflammatory function is not fully understood, previous studies suggested that C-PC promotes the relative expression of antioxidant enzymes and down-regulates the TLR2/MyD88/NF-κB pathway, leading to anti-inflammatory roles. Other studies suggested that C-PC activates the Nrf2 signaling pathway by upregulating the content of Nrf2 and HO-1 in various tissues, thus reducing inflammation. The other mechanism is through the inhibition of NADPH oxidase mimicking the function of biliverdin.

Phycocyanin, a pigment-protein complex derived from Spirulina, has been investigated for its potential therapeutic applications across various medical indications. Studies have suggested that phycocyanin exhibits anticancer properties, including induction of apoptosis, autophagy, and inhibition of angiogenesis, in various cancer cell models, such as breast, liver, lung, colon, leukemia, and bone marrow cancers. Additionally, phycocyanin has been associated with anti-inflammatory and antioxidant effects, which may contribute to its potential role in managing oxidative stress and inflammation-related disorders. Research has also explored its possible hepatoprotective and nephroprotective effects, indicating potential applications in liver and kidney health. Further studies have suggested that phycocyanin could function as a radiosensitizer, potentially enhancing the efficacy of radiation therapy in oncology.

However, the development of phycocyanin into a pharmaceutical formulation presents certain challenges. Phycocyanin is inherently unstable, demonstrating sensitivity to light, temperature, enzymatic degradation, and pH variations, which may affect its bioactivity over time. Additionally, its hydrophilic nature may limit its ability to permeate lipid-based biological membranes, potentially affecting its absorption and systemic bioavailability. SLNs may provide solutions for enhancing the stability and bioavailability of phycocyanin, thereby broadening its potential use in oncology, immunology, metabolic disorders, and organ protection as well as other unexplored conditions.

Since C-PC is sensitive to pH, temperature, and light, and can be easily inactivated during storage, SLNs provide an effective mechanism to deliver this bioactive compound, while protecting C-PC from degradation.

Upon encapsulation into SLNs, the intensely blue-colored C-PC transforms into an off-white to pale yellow color, confirming the effectiveness of lipid encapsulation in masking visible pigmentation. This color modification expands the applicability of phycocyanin beyond traditional blue-colored formulations, allowing for integration into functional foods, cosmetics, and nutraceuticals where color neutrality is preferred. Additionally, while raw C-PC has a distinct, earthy marine odor, the SLN formulation significantly reduces this odor due to the lipid matrix acting as an odor barrier, enhancing its potential for consumer-friendly applications.

Beyond its antioxidant and anti-inflammatory properties, C-PC-loaded SLNs serve as a highly effective natural and sustainable skincare ingredient, capable of penetrating the dermal layers and delivering bioactive nutrients directly to skin cells. This transdermal delivery mechanism supports skin hydration, collagen synthesis, and protection against oxidative stress, making C-PC an ideal bioactive for anti-aging, skin repair, and post-surgical healing formulations. The stabilization of C-PC within SLNs ensures prolonged bioavailability and sustained release, preventing rapid degradation under environmental stressors such as light, heat, and oxidation.

In an embodiment, the bioactive component 100 comprises Vitamin D. Since many elderly people are deficient in vitamin D and certain individuals have trouble absorbing it orally due to gastrointestinal conditions, transdermal delivery may offer an alternative route for supplementation; however, traditional transdermal patches have shown limited efficacy in delivering vitamin D. Using an SLN to encapsulate vitamin D enhances bioavailability and protects the encapsulated material from degradation caused by light, heat, or oxidation. The SLNs provide a gradual release of active ingredients, ensuring a consistent supply of vitamin D over time and reducing the need for frequent dosing. Using a dissolvable microneedle (DMN) patch to deliver the SLNs increases the likelihood that the active ingredients are delivered directly into the dermis, enabling faster and more efficient absorption compared to the passive diffusion mechanism of traditional patches.

Antioxidants and anti-inflammatory compounds are essential for combating oxidative stress and inflammation, both of which contribute to aging, chronic diseases, and various skin conditions. SLNs can encapsulate the following antioxidants and anti-inflammatory compounds:

Phycocyanin, as mentioned above, provides anti-inflammatory benefits for conditions such as inflammatory bowel disease (IBD), joint pain, and skin disorders. Astaxanthin, known for its potent antioxidant properties, protects the skin against UV damage and promotes skin repair and improves joint health. Fucoxanthin supports metabolism and has potential anti-obesity effects. Quercetin, a flavonoid, offers cardiovascular and anti-inflammatory benefits. Silymarin is beneficial for liver health and provides antioxidant protection, while epigallocatechin gallate (EGCG) offers anti-inflammatory and cardiovascular benefits.

Lutein and zeaxanthin are critical for eye health and offer protection against oxidative stress, particularly in the eye. These compounds, when encapsulated in SLNs, provide effective anti-aging and anti-inflammatory action.

Algae-derived bioactives are rich in antioxidants, anti-inflammatory compounds, and other bioactive ingredients that promote skin health, immune function, and general wellness. These bioactives are particularly valuable for their therapeutic effects and their use in nutraceuticals, cosmeceuticals, and pharmaceuticals. Examples of algae-derived bioactives that benefit from SLN encapsulation include:

Spirulina, a blue-green algae, is rich in proteins, vitamins (especially B12), minerals, and antioxidants such as phycocyanin and gamma-linolenic acid (GLA). Chlorella, another algae, is known for its detoxifying properties and high chlorophyll content, which is beneficial for skin health and detoxification. Aphanizomenon flos-aquae (AFA) is rich in proteins, amino acids, and omega-3 fatty acids, which are used for detoxification and immune support. Select species and their bioactive compounds may be incorporated, particularly those with antiviral or protective properties. These include: Griffithsin from red algae (active against SARS, Herpes simplex, and HPV), Cyano-virin from Nostoc ellipsosporum (HIV-1), Scytovirin and Scytonemin from Scytonema (Ebola virus, UV protection), Ca-Spirulan from Spirulina (HIV-1), and lectins from Microcystis (HIV-1), and bioactive compounds from Phormidium tenue, which exhibit antimicrobial and immunomodulatory potential.

Chlorella (Chlorella vulgaris) is a unicellular green microalga belonging to the phylum Chlorophyta, widely recognized for its high nutritional value and therapeutic potential. It is rich in proteins, essential amino acids, vitamins, minerals, and bioactive compounds such as chlorophyll, carotenoids, and polysaccharides. Due to its dense nutrient profile, Chlorella has been extensively studied for its applications in nutraceuticals, functional foods, and therapeutic formulations.

One of the most notable benefits of Chlorella is its ability to detoxify heavy metals and toxins from the body. Its high chlorophyll content and unique cell wall composition allow it to bind and facilitate the excretion of toxic metals such as lead, mercury, and cadmium, making it an effective natural detoxifying agent. Additionally, Chlorella has been shown to enhance immune system function by stimulating the activity of natural killer (NK) cells, macrophages, and cytokine production. These immune-modulating properties contribute to increased resistance against infections and inflammatory conditions, promoting overall immune resilience.

Beyond its immune and detoxification benefits, Chlorella supports metabolic health by regulating cholesterol and blood sugar levels. Research suggests that Chlorella can lower LDL cholesterol and triglycerides while increasing high density lipoprotein (HDL) cholesterol, contributing to cardiovascular health. Furthermore, its bioactive compounds improve glucose metabolism and insulin sensitivity, making it a potential natural intervention for conditions such as diabetes and dyslipidemia. Chlorella also plays a vital role in digestion and gut health, as its fiber and polysaccharides promote the growth of beneficial gut bacteria, support intestinal integrity, and reduce symptoms associated with gastrointestinal disorders like irritable bowel syndrome (IBS) and leaky gut syndrome.

Despite its numerous health benefits, Chlorella faces several challenges that limit its bioavailability and absorption. Its outer cell wall is rigid and indigestible, making it difficult for the human body to fully absorb its nutrients. Additionally, many of its bioactive compounds, including chlorophyll and carotenoids, have poor water solubility, which reduces their uptake in the digestive system. The degradation of these bioactives by stomach acids and digestive enzymes further diminishes its efficacy.

To overcome these limitations, SLNs can be encapsulated and present a promising solution. SLNs can enhance the bioavailability of Chlorella's bioactive compounds (such as chlorophyll, lutein & zeaxanthin, chlorella growth factor (CGF), omega-3 fatty acids (DHA & EPA), sulfated polysaccharides) by improving their solubility and stability, ensuring better absorption in the body. This SLN technology also enables controlled and sustained release of nutrients, prolonging their therapeutic effects. Furthermore, SLNs protect Chlorella's sensitive compounds from degradation by stomach acids and enzymes, preserving their potency. Additionally, SLN formulations can be designed for targeted delivery, optimizing nutrient uptake at specific absorption sites such as the intestines.

Chlorella vulgaris is a powerful natural superfood with significant detoxifying, immune-boosting, metabolic, and gut health benefits. However, its limited bioavailability and absorption hinder its full potential. The use of SLN technology offers a novel and effective approach to overcoming these challenges, enhancing the delivery and efficacy of Chlorella-derived bioactive compounds.

Haematococcus pluvialis is a freshwater microalga belonging to the Chlorophyceae family, widely recognized as the richest natural source of astaxanthin, a powerful carotenoid with exceptional antioxidant properties. The deep red pigment of astaxanthin not only gives Haematococcus pluvialis its characteristic color but also contributes to its extensive applications in nutraceuticals, cosmetics, and pharmaceutical formulations.

Astaxanthin is known for its profound health benefits, particularly in skin health, immune support, and inflammation reduction. As a potent antioxidant, it neutralizes reactive oxygen species (ROS) and protects skin cells from oxidative damage caused by UV radiation and environmental pollutants. This makes it a valuable ingredient in anti-aging skincare products, as it helps to maintain skin elasticity, reduce the appearance of wrinkles, and prevent photoaging. Additionally, astaxanthin has been shown to enhance immune system function by modulating inflammatory responses, reducing excessive oxidative stress, and improving the activity of immune cells. It plays a critical role in supporting overall immunity and protecting against infections. Furthermore, its anti-inflammatory properties extend beyond immune function, as studies have demonstrated its effectiveness in reducing inflammation associated with chronic conditions such as arthritis, cardiovascular diseases, and neurodegenerative disorders.

Despite its extensive health benefits, the bioavailability of astaxanthin from Haematococcus pluvialis remains a challenge. Astaxanthin is a lipophilic compound with poor water solubility, which limits its absorption in living beings. Moreover, it is highly susceptible to degradation when exposed to heat, oxygen, and light, leading to reduced stability and efficacy. Traditional delivery methods, such as soft gel capsules and powdered supplements, often fail to protect astaxanthin from oxidative degradation, resulting in diminished potency over time.

Encapsulation in solid lipid nanoparticles (SLNs) offers an innovative solution to these limitations by enhancing the stability, bioavailability, and controlled release of astaxanthin. SLNs create a protective lipid matrix around astaxanthin molecules, shielding them from oxidative damage and environmental degradation. This encapsulation method also improves the solubility of astaxanthin, ensuring more efficient absorption in the gastrointestinal tract. Furthermore, SLNs provide a controlled and sustained release of astaxanthin, allowing for prolonged circulation in the bloodstream and maximizing its therapeutic effects. By utilizing SLN, astaxanthin can be formulated into more effective and stable nutraceuticals, cosmeceuticals, and pharmaceutical applications.

Nannochloropsis is rich in omega-3 fatty acids (EPA), proteins, and carotenoids, and is often used for cardiovascular health. Nannochloropsis is a genus of unicellular microalgae within the class Eustigmatophyceae, renowned for its rapid growth and high lipid content, particularly its richness in omega-3 polyunsaturated fatty acids (PUFAs) such as eicosapentaenoic acid (EPA). This microalga is also a valuable source of bioactive compounds, including carotenoids such as astaxanthin, canthaxanthin, and zeaxanthin, which are well-documented for their potent antioxidant properties. Due to its exceptional nutrient profile, Nannochloropsis has been extensively studied for its potential applications in nutraceuticals, functional foods, and therapeutic formulations.

One of the key health benefits of Nannochloropsis is its contribution to cardiovascular health, primarily due to its high EPA content. EPA plays a crucial role in reducing inflammation, lowering triglyceride levels, and improving overall lipid metabolism, thereby reducing the risk of cardiovascular diseases. Studies suggest that regular consumption of Nannochloropsis-derived omega-3s can improve vascular function, decrease platelet aggregation, and reduce oxidative stress, which are all critical factors in heart disease prevention.

In addition to its cardiovascular benefits, Nannochloropsis has been shown to enhance immune system function. Research on Nannochloropsis oculata has demonstrated that its supplementation can modulate immune responses by increasing cytokine production and improving lymphocyte activity. These immunomodulatory effects suggest that Nannochloropsis may be beneficial in strengthening the body's defense mechanisms against infections and inflammatory diseases.

Furthermore, the carotenoid content in Nannochloropsis contributes to its strong antioxidant capabilities, helping to neutralize free radicals and reduce oxidative stress. Oxidative damage is a primary contributor to aging and the development of chronic diseases, including neurodegenerative disorders, cardiovascular diseases, and certain types of cancer. By scavenging reactive oxygen species (ROS), the bioactive compounds in Nannochloropsis support cellular health and promote longevity.

Despite its significant health benefits, Nannochloropsis-derived bioactive compounds face challenges related to bioavailability, stability, and effective delivery. The lipophilic nature of EPA and carotenoids makes them poorly soluble in water, limiting their absorption in the gastrointestinal tract. Additionally, these bioactives are highly susceptible to oxidative degradation when exposed to light, heat, and oxygen, which reduces their efficacy over time. Encapsulation within solid lipid nanoparticles (SLNs) presents a promising solution to these limitations. SLNs can protect Nannochloropsis-derived bioactives from environmental degradation, enhance their solubility, and improve their absorption in the body. The lipid matrix of SLNs allows for controlled and sustained release, ensuring prolonged therapeutic effects while increasing bioavailability. By leveraging SLN technology, Nannochloropsis-based formulations can be optimized for use in pharmaceuticals, nutraceuticals, and functional food applications, ultimately maximizing their health benefits and commercial potential.

Schizochytrium is a genus of unicellular marine microalgae within the family Thraustochytriaceae, commonly found in coastal environments. These microalgae are particularly notable for their high content of omega-3 polyunsaturated fatty acids (PUFAs), especially docosahexaenoic acid (DHA), with smaller amounts of eicosapentaenoic acid (EPA). Unlike Nannochloropsis, which is predominantly a source of EPA, Schizochytrium is an excellent natural source of DHA, making it valuable for supplementation in functional foods, nutraceuticals, and pharmaceuticals.

One of the key health benefits of Schizochytrium is its role in cardiovascular health. The high DHA content has been linked to reducing triglyceride levels, lowering blood pressure, and improving overall heart function, thereby decreasing the risk of cardiovascular diseases. Additionally, DHA is essential for neurological health, as it is a critical component of neuronal membranes. Studies suggest that supplementation with Schizochytrium-derived DHA supports cognitive function, enhances brain plasticity, and may help slow cognitive decline associated with aging. Another significant benefit of Schizochytrium is its impact on eye health. DHA is a major structural component of the retina, and adequate intake has been associated with a reduced risk of age-related macular degeneration and improvements in dry eye symptoms.

Furthermore, Schizochytrium exhibits potent anti-inflammatory properties due to its omega-3 fatty acids, which help regulate inflammatory responses. These properties make it a potential therapeutic ingredient for conditions such as arthritis and other chronic inflammatory disorders. The bioactive compounds in Schizochytrium also serve as antioxidants, protecting cells from oxidative stress and contributing to overall cellular health. While both Schizochytrium and Nannochloropsis offer significant health benefits, their omega-3 profiles differ, with Schizochytrium being a superior source of DHA and Nannochloropsis providing higher levels of EPA. Both microalgae play an essential role in supporting cardiovascular function, immune system regulation, and antioxidant activity, making them valuable components in advanced functional nutrition and therapeutic applications.

Fucoidan is a sulfated polysaccharide primarily found in the cell walls of brown seaweeds such as Undaria pinnatifida (wakame), Fucus vesiculosus, and Laminaria japonica. It consists mainly of fucose and sulfate groups, with minor amounts of other sugars, uronic acids, and acetyl groups. Due to its unique structure, fucoidan exhibits a broad range of biological activities, making it a valuable compound in nutraceutical, pharmaceutical, and functional food applications. However, despite its therapeutic potential, its poor bioavailability and susceptibility to enzymatic degradation limit its effectiveness, highlighting the need for advanced delivery technologies such as SLNs.

One of the most well-documented benefits of fucoidan is its ability to modulate the immune system and reduce inflammation. It enhances the activity of immune cells, including macrophages, T cells, and natural killer (NK) cells, strengthening immune responses while inhibiting the production of pro-inflammatory cytokines such as TNF-α, IL-6, and IL-1β. This makes fucoidan a promising natural compound for managing inflammatory diseases such as arthritis and autoimmune conditions. In addition to its immune-modulating effects, fucoidan has demonstrated significant anticancer properties. Several studies indicate that it can inhibit tumor cell proliferation, induce apoptosis (programmed cell death), and suppress angiogenesis, which is the formation of new blood vessels that supply tumors. These mechanisms make fucoidan a potential candidate for integrative cancer therapies.

Fucoidan has also shown potent antiviral activity against influenza, herpes simplex virus (HSV), and human immunodeficiency virus (HIV) by preventing viral adhesion and entry into host cells. Its antimicrobial properties extend to antibacterial and antifungal effects, making it a natural alternative for combating infections. Cardiovascular health is another key area where fucoidan provides benefits. Research suggests that it can lower blood pressure, reduce cholesterol levels, and exhibit anticoagulant activity, which may help prevent blood clot formation. Furthermore, fucoidan plays a role in metabolic health by improving insulin sensitivity and regulating blood sugar levels, making it beneficial for individuals with diabetes or metabolic disorders.

Beyond metabolic and cardiovascular benefits, fucoidan contributes to gastrointestinal health by acting as a prebiotic, supporting the growth of beneficial gut bacteria such as Lactobacillus and Bifidobacterium. It also helps protect the intestinal lining, potentially reducing the risk of leaky gut syndrome and inflammatory bowel diseases like ulcerative colitis and Crohn's disease. Emerging research suggests that fucoidan has neuroprotective effects, particularly in reducing oxidative stress and inflammation in the brain, which may have implications for preventing neurodegenerative conditions such as Alzheimer's and Parkinson's disease. Its antioxidant properties further contribute to anti-aging effects by protecting skin cells from oxidative damage and promoting collagen synthesis.

Despite these extensive health benefits, fucoidan's therapeutic applications are limited due to its low bioavailability, poor solubility, and susceptibility to enzymatic degradation in the gastrointestinal tract. Encapsulation in solid lipid nanoparticles (SLNs) presents a promising solution to these challenges by enhancing stability, protecting fucoidan from environmental degradation, and improving its absorption and controlled release. By utilizing SLN technology, fucoidan's therapeutic effects can be maximized, allowing for more effective applications in pharmaceuticals, nutraceuticals, and cosmeceuticals.

Fucoxanthin is a naturally occurring carotenoid primarily found in brown seaweeds such as Undaria pinnatifida (wakame), Laminaria japonica, and Sargassum species. As a xanthophyll pigment, fucoxanthin plays a crucial role in photosynthesis and provides the distinctive brown coloration of these algae. Unlike other common carotenoids such as β-carotene or lutein, fucoxanthin possesses a unique molecular structure that contributes to its potent antioxidant, anti-inflammatory, and metabolic health benefits. However, despite its significant therapeutic potential, fucoxanthin's poor bioavailability and instability limit its effectiveness, necessitating advanced delivery technologies such as solid lipid nanoparticles (SLNs) to enhance its efficacy.

One of the most widely researched benefits of fucoxanthin is its role in metabolic health, particularly in weight management and obesity reduction. Studies indicate that fucoxanthin promotes fat metabolism by stimulating the expression of uncoupling protein 1 (UCP1) in white adipose tissue, leading to increased thermogenesis and energy expenditure. This mechanism helps reduce body fat accumulation, making fucoxanthin a promising natural compound for managing obesity and metabolic disorders such as type 2 diabetes. Additionally, fucoxanthin has been shown to improve insulin sensitivity and regulate glucose metabolism, further supporting its role in metabolic health.

Beyond its metabolic benefits, fucoxanthin exhibits strong antioxidant and anti-inflammatory properties. As a potent scavenger of reactive oxygen species (ROS), fucoxanthin protects cells from oxidative stress, reducing the risk of chronic diseases such as cardiovascular disorders, neurodegenerative diseases, and cancer. Research suggests that fucoxanthin has anticancer properties, as it can induce apoptosis (programmed cell death) in various cancer cell lines, including breast, lung, prostate, and colon cancer. Its anti-angiogenic effects further inhibit tumor progression by preventing the formation of new blood vessels that supply nutrients to tumors.

Fucoxanthin has also demonstrated significant cardiovascular benefits. Studies suggest that it helps lower LDL cholesterol while increasing HDL cholesterol, contributing to improved lipid profiles and reduced risk of atherosclerosis. Additionally, it has been shown to regulate blood pressure and improve endothelial function, further supporting heart health. Its ability to reduce systemic inflammation makes it beneficial for conditions such as arthritis, inflammatory bowel disease, and autoimmune disorders.

In addition to its systemic health benefits, fucoxanthin has been studied for its role in skin health and anti-aging. As a powerful antioxidant, it protects skin cells from UV-induced oxidative damage and helps prevent premature aging. Studies suggest that fucoxanthin promotes collagen production and improves skin elasticity, making it a valuable ingredient for cosmeceutical applications. Furthermore, its neuroprotective effects have been explored in the context of cognitive health, as it has been shown to reduce inflammation and oxidative damage in neuronal cells, potentially lowering the risk of Alzheimer's and Parkinson's diseases.

Despite these extensive health benefits, fucoxanthin's clinical applications are hindered by its low bioavailability, poor water solubility, and susceptibility to degradation when exposed to heat, light, and oxygen. Encapsulation in solid lipid nanoparticles (SLNs) provides a promising strategy to overcome these challenges. SLNs can enhance fucoxanthin's stability and solubility, protecting it from oxidative degradation while improving absorption in the gastrointestinal tract. Additionally, SLNs allow for controlled and sustained release, ensuring prolonged circulation and maximizing fucoxanthin's therapeutic effects. By incorporating fucoxanthin into SLN-based formulations, its potential applications in pharmaceuticals, nutraceuticals, and cosmeceuticals can be significantly expanded, paving the way for more effective and bioavailable fucoxanthin-based products.

Ecklonia is a genus of kelp belonging to the family Lessoniaceae, with species such as Ecklonia cava predominantly found in the coastal regions of Japan and Korea. These brown algae are particularly rich in phlorotannins, a unique type of polyphenol found exclusively in brown seaweeds. Phlorotannins have been widely studied for their diverse health benefits, including antioxidant, anti-inflammatory, cardiovascular, and metabolic effects.

One of the most notable benefits of Ecklonia cava is its strong antioxidant activity, which is primarily attributed to its high phlorotannin content. These compounds help neutralize free radicals and reduce oxidative stress, a key factor in aging and the development of chronic diseases such as neurodegenerative disorders, cardiovascular disease, and cancer. In addition to its antioxidant effects, Ecklonia cava has demonstrated anti-inflammatory properties, making it a potential therapeutic agent for managing inflammatory conditions such as arthritis and autoimmune diseases.

Research has also highlighted Ecklonia cava's cardiovascular benefits, with studies suggesting that its extracts may improve blood circulation, reduce cholesterol levels, and support overall heart health. By modulating lipid metabolism and improving endothelial function, Ecklonia cava may help lower the risk of atherosclerosis and hypertension. Furthermore, its bioactive compounds have been linked to weight management, as they can inhibit the absorption of fats and carbohydrates, contributing to reduced fat accumulation and improved metabolic health.

Research indicates that Ecklonia cava extracts stimulate the proliferation of human dermal papilla cells (DPCs) and outer root sheath (ORS) cells, both crucial for hair follicle development and cycling. In vitro studies demonstrated that treatment with Ecklonia cava extract led to significant elongation of hair shafts in cultured human hair follicles. Additionally, topical application of the extract on mice promoted the transition of hair follicles from the resting (telogen) phase to the active growth (anagen) phase. These effects are partly attributed to the upregulation of insulin-like growth factor-1 (IGF-1) expression in DPCs, a growth factor known to play a vital role in hair growth regulation.

Despite its promising therapeutic potential, Ecklonia cava's bioavailability is limited due to its poor solubility and rapid degradation in the digestive system. To overcome these challenges, SLNs enhance the stability and absorption of its bioactive compounds. SLNs can protect Ecklonia cava's polyphenols from oxidative degradation, improve their solubility, and allow for sustained release, maximizing their therapeutic effects.

Porphyridium cruentum is a unicellular red microalga belonging to the phylum Rhodophyta, widely recognized for its production of high-value bioactive compounds, including sulfated polysaccharides, phycoerythrin, and polyunsaturated fatty acids (PUFAs). These compounds contribute to P. cruentum's extensive health benefits, making it a promising ingredient in nutraceutical, pharmaceutical, and cosmeceutical applications. Due to its potent antioxidant, anti-inflammatory, and antimicrobial properties, P. cruentum has been extensively studied for its role in promoting overall health and wellness. However, despite its therapeutic potential, its poor bioavailability and susceptibility to environmental degradation present challenges that could be addressed through advanced delivery systems such as solid lipid nanoparticles (SLNs).

One of the key health benefits of P. cruentum lies in its strong antioxidant properties. The high concentration of sulfated polysaccharides and phycoerythrin in this microalga allows it to scavenge free radicals, reducing oxidative stress and protecting cells from DNA damage. This contributes to slowing the aging process and lowering the risk of oxidative stress-related diseases such as cardiovascular disorders, neurodegenerative diseases, and cancer. Additionally, P. cruentum exhibits potent anti-inflammatory effects by inhibiting the production of pro-inflammatory cytokines, including TNF-α and IL-6. This suggests that P. cruentum extracts may be beneficial in managing chronic inflammatory conditions such as arthritis, inflammatory bowel disease (IBD), and metabolic disorders.

Beyond its systemic health benefits, P. cruentum is widely studied for its applications in skincare due to its ability to retain moisture, protect against UV-induced damage, and enhance collagen synthesis. Its sulfated polysaccharides create a protective barrier on the skin, preventing moisture loss while reducing oxidative damage from environmental stressors. This makes P. cruentum a valuable component in anti-aging formulations and wound healing treatments. Furthermore, the polyunsaturated fatty acids (PUFAs) in P. cruentum, such as eicosapentaenoic acid (EPA) and arachidonic acid (AA), play a crucial role in heart health, cholesterol regulation, and inflammation reduction. Research suggests that P. cruentum extracts can help lower LDL cholesterol levels, prevent arterial plaque formation, and improve overall lipid metabolism, thereby reducing the risk of cardiovascular disease.

In addition to its cardiovascular benefits, P. cruentum exhibits significant antimicrobial and immune-boosting effects. The sulfated polysaccharides derived from this microalga have demonstrated antiviral, antibacterial, and antifungal properties, inhibiting viral adhesion and replication. These properties make P. cruentum a potential candidate for antiviral therapies against influenza, herpes, and other infections. Moreover, research suggests that P. cruentum extracts enhance immune function by stimulating macrophage and lymphocyte activity, thereby strengthening the body's ability to fight infections. Emerging studies also highlight its neuroprotective potential, as the antioxidants and PUFAs from P. cruentum have been shown to reduce neuroinflammation and oxidative damage in the brain, potentially lowering the risk of neurodegenerative diseases such as Alzheimer's and Parkinson's.

Despite these extensive health benefits, P. cruentum's bioactive compounds face challenges related to stability, solubility, and bioavailability. Sulfated polysaccharides and PUFAs are highly susceptible to degradation in the digestive system and when exposed to environmental factors such as heat and oxidation. Encapsulation into solid lipid nanoparticles (SLNs) is an effective solution by enhancing stability, controlled release, and bioavailability. SLNs protect bioactive compounds from enzymatic degradation, improve gastrointestinal absorption, and allow for sustained delivery, thereby increasing the therapeutic potential of P. cruentum extracts.

Tisochrysis lutea is a unicellular golden-brown microalga belonging to the family Isochrysidaceae within the division Haptophyta. One of the most notable features of T. lutea is its high content of polyunsaturated fatty acids (PUFAs), particularly docosahexaenoic acid (DHA), stearidonic acid, and alpha-linolenic acid. These omega-3 fatty acids are essential for human health, contributing to cardiovascular health, cognitive function, and anti-inflammatory responses. The presence of these PUFAs makes T. lutea a valuable candidate for nutraceutical products aimed at supplementing omega-3 intake.

In addition to its lipid profile, T. lutea is rich in pigments such as fucoxanthin, a carotenoid with potent antioxidant properties. Fucoxanthin has been associated with various health benefits, including anti-inflammatory effects and potential protective roles against metabolic disorders. The amphiphilic nature of fucoxanthin allows it to integrate effectively into biological membranes, enhancing its bioavailability and efficacy.

The unique composition of T. lutea extends its applications beyond human nutrition. It is extensively utilized in aquaculture as a live feed for oyster and shrimp larvae, owing to its rapid growth rate and high nutritional value. The microalga's adaptability to various physico-chemical conditions and its rich PUFA content make it an ideal feed, promoting healthy development in marine larvae.

Recent studies have explored the potential health benefits of T. lutea in mammalian models. For instance, supplementation with T. lutea in high-fat diet-induced obese Wistar rats resulted in reduced body weight, improved lipid profiles, and decreased markers of inflammation. These findings suggest that T. lutea may offer protective effects against metabolic disorders associated with obesity.

Furthermore, the anti-inflammatory properties of T. lutea have been investigated in ocular health. In vitro studies demonstrated that T. lutea extracts could inhibit pathways associated with inflammation in human retinal epithelial cells. In vivo experiments indicated that T. lutea supplementation increased tear production and reduced corneal damage in models of dry eye syndrome, highlighting its potential therapeutic applications in eye health. Its high content of essential fatty acids, valuable pigments, and adaptability to diverse environments highlights its potential as a sustainable resource for various applications.

SLN fabrication of violaxanthin, neoxanthin, alpha-linolenic acid (ALA), stearidonic acid (SDA), laminarin, ulvan, spirulan, lectins, and rhamnan sulfate can enhance the stability, bioavailability, controlled release, and therapeutic efficacy of these bioactive compounds in addition to any algae strain and their bioactive compounds that currently exist or have yet to be discovered.

It is to be understood that the above list of bioactive compounds is not meant to be limiting, and that the present invention may be used with a wide range of bioactive compounds. Some compounds (e.g. proteins) may be sensitive to the temperature or sonication conditions and thus would not be suitable for encapsulation in an SLN using conventional high temperature or high energy methods. However, any compound that can withstand the required temperature and sonication and can be nanosized to a usable particle size is suitable for the present invention. Alternative SLN production techniques, including cold homogenization, solvent evaporation, and microfluidization, can be used to minimize heat exposure and preserve the structural integrity of heat-sensitive compounds. Additionally, selecting lipids with lower melting points can significantly reduce the required processing temperature, thereby protecting encapsulated proteins from thermal degradation.

For example, oleic acid (melting point 13.4° C.) remains liquid at room temperature and can serve as a structural modifier in lipid blends rather than as a primary solid lipid. Cocoa butter (melting point 34-38° C.) remains solid at room temperature and can be combined with other lipids to create a stable SLN formulation while lowering processing temperatures. Alternative lipids such as Precirol® ATO 5 or Compritol® 888 ATO may be used in combination to achieve the desired stability and encapsulation efficiency.

Sonication, a common SLN processing step, can generate localized high temperatures that may degrade sensitive proteins. To mitigate this, cold homogenization or solvent diffusion techniques can be used to produce SLNs under lower energy and temperature conditions. Additionally, stabilizers such as phospholipids, polysorbates, or sugars may be incorporated to protect proteins from shear stress or solvent exposure.

Furthermore, other techniques that reduce heat exposure to proteins during SLN fabrication can also be employed, including emerging or yet to be discovered methods that further optimize the encapsulation of temperature-sensitive bioactives. By refining lipid selection, encapsulation conditions, and processing techniques, SLNs can be effectively adapted to preserve protein integrity while maintaining nanoparticle stability and therapeutic efficacy.

A surfactant 120 is then used to stabilize the nanoparticle formulation during manufacture. The surfactant may be a natural surfactant such as lecithin, saponins, gum Arabic, decyl glucoside; a non-ionic surfactant such as Poloxamer 407, Tween 80, or Span 20; an anionic surfactant with a pharmaceutical grade safety profile such as sodium stearoyl lactylate; or a cationic surfactant such as benzalkonium chloride, cetylpyridinium chloride, and cetyltrimethylammonium bromide (CTAB). Anionic surfactants, such as sodium lauryl sulfate (SLS) and docusate sodium (dioctyl sodium sulfosuccinate) can also be used. The choice of surfactant depends on the physicochemical properties of the nanoparticle system, intended application, desired zeta potential, and biocompatibility considerations.

The surfactant is added during the manufacturing process as will be described below.

While palmitic acid is a naturally occurring lipid found in palm oil, Poloxamer-407 is a synthetic surfactant. Poloxamer-407 is generally regarded as safe and is approved by the US FDA for pharmaceutical applications, but the fact remains that it is a synthetic compound. Thus, in an aspect of the present invention, SLNs are fabricated using palmitic acid [or other natural lipid(s)] as the natural lipid and decyl glucoside (or other natural surfactant(s) as the natural surfactant. This is important for living beings with allergies or sensitivities to synthetic compounds. Decyl glucoside, obtained from 100% renewable raw materials, is derived from the reaction between glucose and the fatty acid alcohol decanol derived from coconut. Decyl glucoside is an effective surfactant. For instance, three formulations using varying percentages of decyl glucoside are used to encapsulate green tea extract in a nano-structured lipid carrier through a high shear homogenization. These particles range from 295 to 472 nm in particle size with a PDI of 0.23 to 0.26, demonstrating that decyl glucoside is a viable alternative for naturally derived surfactant fabrication. In some embodiments of fabrication processes in accordance with the present invention, decyl glucoside is used in combination with palmitic acid, following the previously described procedure. As an example, a combination of a non-ionic surfactant (e.g., Tween 80 or Poloxamer 407) with an ionic surfactant (e.g., sodium lauryl sulfate or cetyltrimethylammonium bromide) is commonly used to optimize nanoparticle formulation.

In an embodiment, a mixture of two or more surfactants may be used to enhance the stability, encapsulation efficiency, and loading capacity of SLNs. The combination of surfactants optimizes interfacial tension reduction, improves nanoparticle dispersion, and prevents aggregation, leading to a more stable formulation with controlled drug release. Suitable surfactant combinations include both ionic and non-ionic surfactants or two non-ionic surfactants with varying hydrophilic-lipophilic balance (HLB) values to achieve optimal stability and bioavailability. For example, a combination of Poloxamer 188 (Pluronic F68) and soy lecithin can be used, where Poloxamer 188 provides steric stabilization to prevent particle aggregation, and soy lecithin, a natural phospholipid, enhances lipid membrane integrity and emulsification, improving encapsulation efficiency and drug retention. Another suitable combination is Tween 80 (Polysorbate 80) and Span 60 (Sorbitan Monostearate), where Tween 80, a high-HLB non-ionic surfactant, facilitates nanoparticle dispersion in aqueous media, and Span 60, a low-HLB surfactant, strengthens the lipid core and improves drug retention, resulting in enhanced stability and controlled drug release. In another embodiment, sodium lauryl sulfate (SLS) and Poloxamer 407 (Pluronic F127) may be used to enhance dispersion and steric stabilization, wherein SLS provides electrostatic stabilization by reducing particle aggregation and Poloxamer 407 contributes steric stabilization for long-term formulation stability. However, in pharmaceutical formulations where SLS toxicity may be a concern, sodium deoxycholate may be used as a safer alternative. The selection of surfactant combinations is optimized based on the desired application, lipid composition, and intended release kinetics to improve the overall efficacy and performance of SLN formulations for pharmaceutical, nutraceutical, cosmetic, and functional food applications.

In certain embodiments, more than two surfactants may be used in combination to further improve stability, bioavailability, and bioactive compound loading capacity. A ternary surfactant system may include Poloxamer 188, soy lecithin, and Tween 80, where Poloxamer 188 provides steric stabilization, soy lecithin enhances lipid membrane integrity, and Tween 80 improves emulsification and dispersion. Another example of a three-surfactant system includes Span 60, Tween 80, and sodium deoxycholate, where Span 60 stabilizes the lipid phase, Tween 80 enhances aqueous dispersibility, and sodium deoxycholate provides additional electrostatic stabilization. The use of multiple surfactants allows for tailored interfacial properties, which can be particularly useful for multi-drug delivery systems, targeted release applications, and formulations requiring prolonged stability under varying physiological conditions.

It is understood that the surfactants used in SLN formulations are not limited to the combinations listed herein, and various other surfactant systems, including those not yet discovered, may be employed to optimize the formulation. The selection of surfactants and their ratios may be modified depending on the specific lipid composition, solubility of the bioactive compound, and intended application in pharmaceutical, nutraceutical, cosmetic, and functional food formulations.

### Method of Manufacture

The fabrication of solid lipid nanoparticles (SLNs) can be achieved through various methods, each providing advantages and limitations depending on the desired particle size, PDI, zeta potential, EE, stability, and scalability. The method of the present invention offers a much higher encapsulation efficiency (EE) than prior-art methods, making it much more useful. The SLN manufacture methods of the present invention, as described below, can reach a 99.93% encapsulation efficiency according to experimental testing.

In some embodiments, high-pressure homogenization (HPH) involves subjecting lipid-drug mixtures to extreme pressure (100-2000 bar) to create nanosized particles. This method includes hot HPH, where the drug is mixed with molten lipids before homogenization and cooling to form SLNs, and cold HPH, where the lipid-drug mixture is rapidly cooled using liquid nitrogen or dry ice, followed by homogenization under cold conditions. Cold HPH is particularly beneficial for thermosensitive drugs but generally results in larger particle sizes compared to hot HPH. Another commonly used technique, high-speed stirring and ultra-sonication, utilizes high shear forces and cavitation to break down lipid particles into the nanometer range. This method is accessible, but probe ultrasonication can result in metal contamination from the sonicator probe and expose drug compounds to prolonged high temperatures.

The microemulsion method produces SLNs through the formation of a thermodynamically stable microemulsion at elevated temperatures, which is then rapidly cooled to precipitate lipid nanoparticles. While this method ensures high reproducibility, it requires large volumes of surfactants and water, making it less practical for large-scale production. Solvent emulsification-diffusion and solvent emulsification-evaporation methods involve dissolving lipids and bioactive compounds in organic solvents before emulsification into an aqueous phase, followed by either solvent diffusion or evaporation to induce nanoparticle formation. These methods allow precise control over particle size and are particularly suitable for hydrophilic and hydrophobic bioactive compounds. However, the requirement for organic solvents necessitates additional purification steps to remove solvent residues, which may limit their applicability in pharmaceutical formulations.

For bioactive compounds with water solubility challenges, the double emulsion method is frequently employed, utilizing a water-in-oil-in-water (w/o/w) system where the drug is first emulsified within a lipid phase, followed by re-emulsification into an aqueous phase. While this method is particularly effective for encapsulating proteins and peptides, it often results in a low EE and larger particle sizes. Another widely studied technique, the phase inversion temperature (PIT) method, relies on temperature-induced phase transitions of surfactants, allowing the transformation of a water-in-oil (w/o) emulsion into an oil-in-water (o/w) nanoemulsion through rapid cooling. This energy-efficient, solvent-free approach is advantageous for stabilizing nanoemulsions, though it may require multiple temperature cycles to achieve optimal results.

The membrane contactor method is an emerging technique that involves pressing molten lipid through membrane pores while an aqueous surfactant phase circulates on the other side, forming uniform SLNs. This method allows precise size control and scalability but is prone to membrane clogging. Supercritical fluid-based methods, such as supercritical CO₂-assisted precipitation, offer an environmentally friendly alternative by rapidly removing solvents and forming highly uniform nanoparticles. However, the requirement for specialized high-cost equipment limits its widespread industrial application. The coacervation method, which relies on pH-induced precipitation of lipid salts, provides a solvent-free alternative but is limited to lipids capable of forming alkaline salts and is unsuitable for pH-sensitive drugs.

The solvent injection method involves injecting a lipid-solvent mixture into an aqueous surfactant phase under constant stirring, causing rapid solvent diffusion and lipid precipitation into SLNs. This method is simple, scalable, and does not require high-pressure equipment, making it particularly suitable for laboratory-scale production. However, solvent removal is necessary, and additional post-processing may be required to achieve the desired particle characteristics. Other modifications of solvent injection include microfluidic-based techniques, which enhance control over SLN formation by precisely mixing lipid and aqueous phases at the microscale, leading to more uniform particle distributions. Electrospray methods have also been explored, utilizing an electric field to disperse lipid droplets into nanoparticles, potentially enabling more efficient drug loading and improved stability.

The solvent diffusion method is a variation of the solvent injection method that enables controlled precipitation of lipids in an aqueous environment, ensuring uniform particle size and high encapsulation efficiency. In this method, the lipid and bioactive compound are first dissolved in a partially water-miscible organic solvent such as ethanol, acetone, or ethyl acetate. This lipid phase is then introduced gradually into an aqueous phase containing a surfactant under mechanical stirring or homogenization. As the organic solvent diffuses slowly into the aqueous phase, lipid crystallization occurs, forming nanoparticles in a controlled manner. Unlike the solvent injection method, where the organic phase is forcefully injected into the aqueous phase, causing rapid precipitation, the solvent diffusion method allows for gradual solvent exchange, leading to more uniform nanoparticles with improved stability and lower solvent residue. The solvent diffusion technique provides greater control over particle size and can be optimized for specific drug delivery applications, making it a valuable alternative for SLN production. Both methods share a common principle of organic solvent dispersion into an aqueous medium, but the choice between them depends on desired particle characteristics, scalability, and solvent removal efficiency. The solvent diffusion method may be used alone or in combination with solvent injection to enhance nanoparticle formation, and future variations or yet-to-be-discovered methods that facilitate controlled lipid precipitation may also be employed for improved SLN fabrication.

Beyond these established methods, it is expected that yet-to-be-discovered techniques or equivalent fabrication methods may be developed in the future to enhance SLN production, optimize encapsulation efficiency, and ensure sustainability. Advances in self-assembling lipid systems, nanocarrier hybrids, bioengineered lipid matrices, and AI-driven process optimization may yield novel SLN fabrication techniques with improved scalability and environmental compatibility. The potential for hybrid approaches that combine elements of multiple existing methods may also be utilized to enhance particle stability, controlled drug release, and increased bioavailability.

The characterization of SLNs is essential for ensuring batch-to-batch reproducibility, safety, and therapeutic efficacy, requiring comprehensive physicochemical analysis, lipid composition verification, bioactive stability assessments, and process optimization. All analytical methods referenced herein include current gold-standard techniques or any equivalent method known in the field or developed in the future, allowing for future advancements in testing methodologies and ensuring adaptability to new technologies.

SLNs are characterized by particle size distribution, polydispersity index (PDI), surface charge (zeta potential), encapsulation efficiency (EE %), and loading capacity (LC %), all of which influence their stability, bioavailability, and controlled release properties. Particle size distribution is measured using dynamic light scattering (DLS), nanoparticle tracking analysis (NTA), or any equivalent method, ensuring SLN sizes range from 10 nm approximately 1000 nm. PDI, measured by DLS or NTA indicates uniformity of particle size distribution. Zeta potential, measured using dynamic light scattering (DLS), electrophoretic light scattering (ELS), or an equivalent method, must have an absolute value of ≥25 mV, ensuring sufficient electrostatic repulsion to maintain colloidal stability. Encapsulation efficiency (EE %) and loading capacity (LC %) are measured using UV-Vis Spectroscopy, high performance liquid chromatography, or liquid chromatography mass spectrometry (LC-MS), amino acid analysis (AAA), or any equivalent method, with target values defined for each bioactive compound.

SLNs are composed of biocompatible lipids and surfactants that influence stability, bioavailability, and release kinetics. Lipid characterization is performed using Fourier transform infrared spectroscopy (FTIR), gas chromatography-mass spectrometry (GC-MS), or HPLC, or any equivalent method, to confirm the presence of lipids such as palmitic acid, stearic acid, triglycerides, and natural oils. The melting point range of lipids (e.g., cocoa butter ˜34° C., stearic acid ˜69° C.) is documented to ensure thermal stability and appropriate formulation selection. Surfactant selection and concentration are optimized through surface tension studies, ensuring effective emulsification and biocompatibility. SLNs incorporate natural surfactants such as lecithin, saponins, gum Arabic, and decyl glucoside, as well as non-ionic surfactants such as Poloxamers, Tween, and Span series, ensuring stable formulations suitable for pharmaceutical and nutraceutical applications.

Bioactive compound stability and bioavailability assessments ensure optimal retention of functional properties in lipid-based SLNs. Solubility and partition coefficients between lipid and aqueous phases are determined to assess compatibility with the SLN core. Structural integrity of the molecules is confirmed using Fourier Transform Infrared (FTIR) spectroscopy differential scanning calorimetry (DSC), or UV-Vis spectroscopy, or any equivalent method to evaluate structural integrity. For proteins and enzymes, enzyme activity assays (e.g., C-phycocyanin fluorescence stability) are performed to ensure encapsulated bioactives remain functionally intact. Controlled release profiles are established through in vitro dissolution studies in simulated body fluids, with target release times optimized for specific therapeutic applications.

Optimizing the fabrication process ensures high encapsulation efficiency, reproducibility, and large-scale manufacturing. Solvent selection, including water, ethanol, or pharmaceutically acceptable solvents, is guided by miscibility and volatility data. The bioactive concentration in the bioactive phase is targeted between 5-50 mg/mL, adjusted based on compound solubility. Lipid concentration in the lipid phase is optimized within 1-20% w/w, ensuring efficient emulsification. The ratio of the bioactive phase to the lipid phase is maintained at 1:2 to 1:10, depending on the desired mean diameter and PDI of the SLNs. A surfactant solution (1-10% in water) is then mixed to the lipid phase at 1-10% w/w of the lipid phase to ensure colloidal stability and prevent aggregation. Sonication and heating parameters are optimized, with heating at up to 70° C. for 5-10 minutes, depending on the lipid melting point, and sonication at 20-40 KHz frequency, 10-60% amplitude, for 2-5 minutes, ensuring nano-scale particle formation. An additional pre-sonication step of the bioactive phase in 15-second intervals until a particle size of <100 nm is achieved may be employed to further refine SLN dispersion.

Stability testing ensures extended shelf-life and real-world durability. Physical and chemical stability assessments include tracking SLN structure over time using TEM or similar analysis at 0, 1, 3, 6, and 12 months or alternative intervals appropriate for determining shelf life or durability. Storage conditions at 4° C., room temperature, and 40° C. determine formulation robustness across different conditions. Freeze-thaw cycles assess the risk of aggregation or phase separation, ensuring SLN formulations remain stable under fluctuating temperatures. Oxidation and hydrolysis resistance are evaluated using peroxide value tests and degradation product detection via HPLC or LC-MS, or any equivalent method to maintain product integrity over time.

Biocompatibility and safety testing ensure SLN formulations meet pharmaceutical and regulatory safety standards. Cytotoxicity assessments, including MTT and LDH assays in cell cultures, evaluate cellular safety, ensuring SLN formulations do not induce toxicity. For transdermal applications, skin irritation and sensitization studies, such as the OECD 439 assay or human patch tests or equivalent test, confirm biocompatibility. Oral and inhalation toxicity studies assess acute and sub-chronic effects in animal models to ensure systemic safety for food, supplement, and pharmaceutical use. For ophthalmic formulations, in vitro corneal toxicity and HET-CAM tests ensure SLN safety for eye applications.

Application-specific testing confirms SLN functionality across diverse delivery systems. Transdermal microneedle patches are tested for dissolution time in simulated skin conditions, drug permeation via Franz diffusion cells, and mechanical integrity of microneedles. Skin cream formulations are evaluated for SLN stability in emulsions over time and skin penetration efficiency through tape-stripping or confocal microscopy. Food incorporation studies assess sensory impact, including odor, taste, and texture modifications, while interaction tests ensure SLN stability in acidic environments. Ophthalmic formulations undergo pH, osmolarity, sterility, and permeability testing, ensuring suitability for eye drop applications. Oral medications containing SLNs are subjected to simulated gastric and intestinal digestion stability tests, as well as bioavailability studies in animal models to determine absorption efficiency.

Regulatory and quality control specifications ensure SLN formulations comply with pharmaceutical, nutraceutical, and food safety standards. All lipids and surfactants are documented for GRAS (Generally Recognized as Safe) status, aligning with FDA, EMA, or other regulatory agency guidelines. Analytical quality control methods, such as HPLC or LC-MS for content uniformity, FTIR and DSC for molecular stability, and microbial limit testing per USP <61>, are used to ensure compliance with pharmaceutical standards. USP <61> is a microbial enumeration test that determines the presence and quantity of microorganisms in non-sterile products, such as pharmaceuticals, nutraceuticals, and cosmetics. The tests allow for the quantitative enumeration of mesophilic bacteria and fungi capable of growing under aerobic conditions.

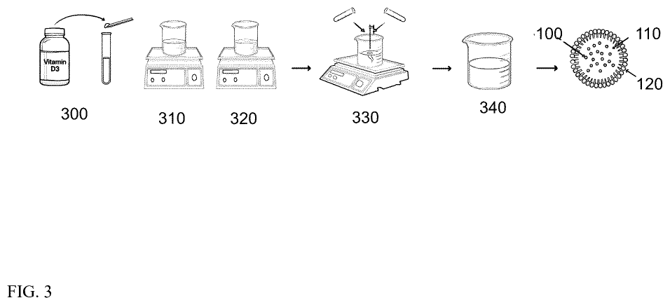

FIG. 3 shows an embodiment of the manufacturing method of the present invention, using vitamin D as an example bioactive compound. It is to be understood that the invention is not limited to that compound. As shown, an embodiment of the method of making a SLN composition can comprise dissolving 300 the bioactive compound in distilled water, ethanol, or any other suitable solvent, such as methanol, acetone, or isopropanol, to create a bioactive phase. The concentration of the bioactive ingredient in the bioactive phase is preferably between 5 and 50 mg/mL. Then, a lipid phase is prepared 310 comprising a lipid. The lipid could be a natural lipid, semi-synthetic, or synthetic lipid as stated above. The lipid is dissolved in a solvent such as ethanol or a pharmaceutically acceptable solvent. The concentration of lipid is preferably 1.0 mg/mL to about 2.0 mg/mL w/w. A surfactant solution 320 is also prepared.

The bioactive phase and the lipid phase are then mixed at a ratio ranging from 1:2 to 1:10, depending on the properties of the ingredients. A surfactant is added to this mixture at a concentration of 1-10% w/w, relative to the combined weight of the bioactive and lipid phases. The mixing step is 330 in FIG. 3. The surfactant may be a natural surfactant, such as lecithin, saponins, gum arabic, decyl glucoside, or Quillaja extract; a biodegradable non-ionic surfactant, such as Poloxamer 407, Tween 80, or Span 20; an anionic surfactant with a pharmaceutical-grade safety profile, such as sodium stearoyl lactylate or cetyltrimethylammonium bromide (CTAB); or an amphiphilic lipid, such as phosphatidylcholine, phosphatidylethanolamine, or glyceryl behenate.

The mixture is then heated to at least 38.4° C. for at least 1 minute and then sonicated 340 at a frequency of 20-40 kHz for 2-5 minutes. This heat-sonication method tends to achieve a mean particle size of less than 200 nm, a polydispersity index (PDI) of less than 0.30, and a zeta potential exceeding+25 mV.

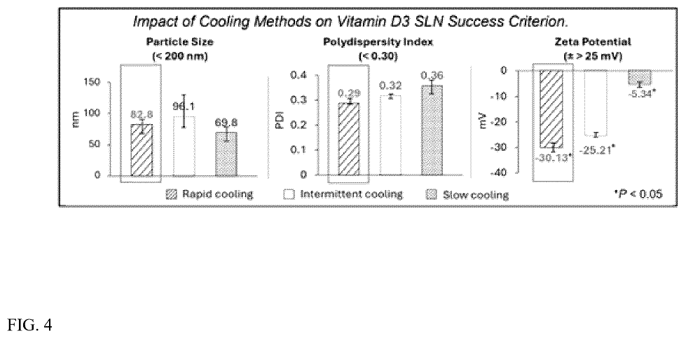

After ultrasonication, three cooling methods are tested to evaluate vitamin D3 SLN properties, as the cooling rate during the fabrication of SLNs significantly influences particle size, zeta potential, and encapsulation efficiency. The ultrasonicated vitamin D3 lipid and surfactant phases underwent rapid cooling (RC, 2° C.), intermittent cooling (IC, 15° C.), or slow cooling (SC, 23° C.). Results of each cooling method on D3 SLN success criterion are summarized in FIG. 4. Each cooling method produced a vitamin D3 SLN with a particle size <200 nm, below the criteria for incorporation into the epidermis. The mean diameter of the vitamin D3 SLNs was: RC, 82.8±8.4 nm; IC, 96.1±30.6 nm; and SC, 69.8±9.2 nm. These values were not significantly different from each other. The PDI of the non-encapsulated vitamin D3 in solution was 0.10±0.01%, indicating a highly homogenous distribution of uniform particles. After vitamin D3 SLN fabrication, PDI of the SLNs was: RC, 0.29±0.05; IC, 0.32±0.02; and SC, 0.36±0.7 (FIG. 3). Additionally, the zeta potential was: RC, −30.13±2.08 mV; IC, −25.22±0.26 mV; and SC, −5.34±0.15 mV, P<0.05 (FIG. 3). While the mean diameters did not significantly differ across cooling methods, PDI and zeta potential indicate that the rapid cooling encapsulation method produces vitamin D3 SLNs that best meet our SLN fabrication criteria.

The mixture is then cooled and the SLNs are dried. After that, they are ready to be used in various formulations as desired.

SLN particle size is influenced by lipid concentration, surfactant percentage, and sonication duration. Adjustments to these parameters are employed to ensure fabrication of SLNs with mean particle size of less than 200 nm. In the event that the absolute value of the zeta potential of the nanoaggregation sized bioactive compound is less than the target of 25 mv, in some embodiments, the zeta potential is further decreased by sonicating the bioactive compound in macromolecular substances such as albumin, dextran, chitosan, hyaluronic acid, casein, pectin, gum arabic, agarose, carrageenan, and gelatin. Also, sonication may be applied to reduce particle size and enhance dispersion and further decreases in zeta potential can be achieved by using an anionic surfactant in the fabrication process.

Lipid concentration and surfactant percentage impact the mean particle size of the SLNs, and a 1.5 mg/mL lipid concentration with 1% surfactant yields SLNs with a mean particle size of 103.4 nm. In order to produce SLNs smaller than 200 nm, this model is adjusted by keeping the lipid concentration at 1.5 mg/mL while increasing the surfactant to 10%.

In an embodiment, if SLN diameter needs to be reduced to a smaller range of 100-200 nm, the following fabrication modifications could be made, each of which independently reduces SLN diameter: 1) reduce viscosity by using a lower melting point lipid than palmitic acid (63° C.), such as lauric acid (44° C.) or by incorporating a lipid blend with a lower solidification; 2) reduce the interfacial tension by increasing the surfactant concentration, thereby increasing the surfactant to lipid ratio or by using a combination of surfactants (e.g., non-ionic+ionic); 3) increase cavitation by increasing ultrasonication duration and/or power, and 4) employ rapid cooling (2° C.) immediately following ultrasonication. Additional modifications include optimizing the lipid-to-drug ratio to help balance crystallinity and prevent excessive lipid content, which can contribute to larger particle formation. Using a co-surfactant system, such as a combination of Tween 80 and Span 20, further enhances emulsification efficiency and ensures uniform size distribution. Modifying the homogenization process, including applying higher-pressure homogenization cycles (e.g., >1000 bar) or multiple passes, can refine nanoparticle size. Adjusting lipid composition by selecting solid lipids with lower melting points, such as glyceryl monostearate, stearic acid, or lauric acid, can facilitate smaller particle formation while maintaining SLN integrity. Incorporating hydrophilic stabilizers, such as polyethylene glycol (PEG) or Pluronic F68, aids in preventing nanoparticle aggregation and reduces polydispersity. Microfluidization can be utilized as an alternative to ultrasonication to ensure uniform energy input, leading to more precise control over nanoparticle size and distribution.

If necessary, additional modifications in SLN fabrication may be employed, such as coating nanoparticles with chitosan or other biopolymers to enhance stability, surface charge, and bioadhesion. Furthermore, polydispersity index (PDI) and zeta potential (ZP) can be modulated using cationic surfactants (e.g., cetyltrimethylammonium bromide, chitosan) or anionic surfactants (e.g., sodium lauryl sulfate, docusate sodium), depending on the desired surface charge properties for stability and cellular uptake. Additionally, based upon the desired SLN diameter and PDI, rapid cooling (2-4 degrees C.), intermittent cooling (5-15 degrees C.) and slow cooling (19-22 degrees C.) may be employed.

In some embodiments, if the bioactive compound has a large particle size, it may be necessary to sonicate the bioactive phase of bioactive compound before encapsulation. This is true for Spirulina, as well as for many other bioactive compounds. In an embodiment, the method may further comprise sonicating the bioactive phase prior to mixing to reduce particle aggregation, where sonication is performed if the bioactive compound exhibits a mean particle size greater than 100 nm or has a tendency to aggregate. The frequency of sonication could be 1-100 W, 10-60% amplitude for 1 to 15 minutes.

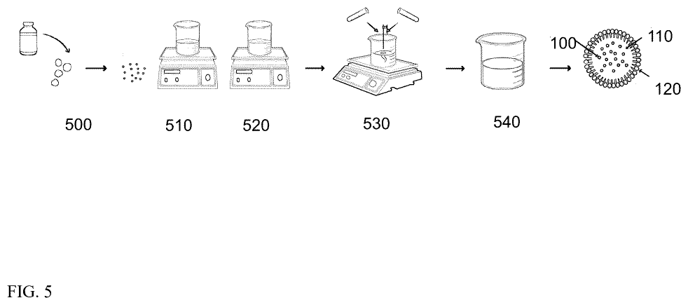

FIG. 5 shows an embodiment of a method for making Spirulina nanoparticles. First, the Spirulina solution is sonicated 500 to reduce particle size. Then, a lipid is dissolved in EtOH 510 and a surfactant is dissolved in dH₂O 520. The nano-sized Spirulina is then mixed 530 with the lipid and surfactant solutions. The mixture is then sonicated and cooled 540, resulting in Spirulina SLNs.

In an embodiment, the sonication could be performed for multiple cycles and durations, depending on the properties of the bioactive compound being encapsulated. In an embodiment, the method may further comprise sonicating the bioactive phase prior to mixing to reduce particle aggregation, where sonication is performed if the bioactive compound exhibits a mean particle size greater than 100 nm or has a tendency to aggregate. Sonication parameters may be optimized based on the physicochemical properties of the bioactive compound, lipid composition, and dispersion medium. In certain embodiments, probe sonication may be performed at a power range of 20 W to 80 W, with an amplitude of 10% to 60%, for 1 to 10 minutes in pulse mode (e.g., 30 seconds on, 10 seconds off) to prevent excessive heat buildup that could degrade thermosensitive bioactive compounds. The specific conditions may be adjusted based on the solubility, crystallinity, and aggregation tendency of the bioactive compound. For instance, hydrophobic compounds such as curcumin, fucoxanthin, and resveratrol may require higher power settings (e.g., 40 W, 30% amplitude, 5 minutes) to achieve effective dispersion and encapsulation within the lipid matrix. Conversely, hydrophilic compounds such as vitamin C, C-phycocyanin, and polyphenols may require milder conditions (e.g., 30 W, 25% amplitude, 3 minutes) to prevent degradation while maintaining uniform dispersion. Amphiphilic compounds, including peptides, phospholipids, and sterols, may benefit from moderate sonication settings (e.g., 50 W, 40% amplitude, 6 minutes, pulse mode) to enhance lipid interaction and improve encapsulation efficiency. In some embodiments, multi-cycle sonication may be performed to further reduce particle size and improve stability, particularly for highly crystalline or poorly soluble bioactives. For example, a two-stage sonication process may involve an initial high-energy burst followed by a secondary lower-intensity cycle (e.g., 5 minutes at 40% amplitude, followed by a 2-minute cooling period, then 3 minutes at 30% amplitude). The method may also include cooling intervals to prevent thermal degradation of the lipid matrix and active compound. It is understood that sonication parameters may be further refined depending on the specific bioactive compound, lipid composition, and solvent system used, and that additional combinations, including those not explicitly described herein, may be employed to achieve optimal formulation properties.

This sonication step is necessary for compounds that have larger than >350 nm particle size, such as Spirulina. For example, to create SLNs containing whole Spirulina, Spirulina powder is sonicated in 15 second intervals at 35% of maximum sonication amplitude. Further reductions in Spirulina nanoparticle size could be obtained by performing continued sonication. This sonication step is also applicable for other bioactive compounds that are >350 nm to reduce particle size prior to SLN fabrication to ensure homogeneity of particle size.

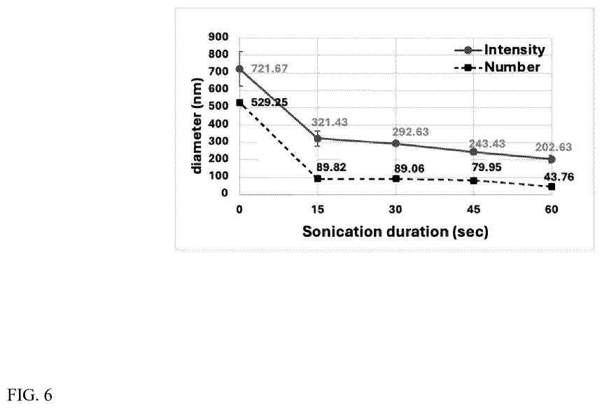

Experimentally, at a 10 mg/mL Spirulina concentration, as shown in FIG. 6, sonication successfully reduced nanosized Spirulina particles from 529.25±52.05 nm to 43.76±21.06 nm after four 15-second sonications. The unsonicated Spirulina solution had a PDI of 0.07±0.22, below the threshold of 0.30 after 30 seconds of vortexing at high speed. Following each of the four 15-second sonications, the PDI remained below 0.30 (0.25±0.82; 0.25±0.22; 0.25±0.19; 0.27±0.12, respectively) with no significant difference between groups (P=0.36). The zeta potential of the nano-sized Spirulina was −22.27±0.43 mV. While the absolute value of the zeta potential of the nano-sized Spirulina is less than the target of 25 mV, the zeta potential may be further decreased by sonicating the Spirulina in macromolecular substances such as albumin, Dextran, polyethylene glycol, or through the incorporation of an anionic surfactant in the fabrication process.

Thus, Spirulina for encapsulation in SLNs can be provided at the desired particle size while preserving the bioactivity of major components.

Following sonication, certain bioactive compounds such as Chlorella vulgaris may contain residual large particulate matter that could interfere with the uniformity and stability of subsequent nanoparticle formulations. To enhance homogeneity and ensure optimal encapsulation efficiency into SLNs, the sonicated mixture is subjected to a filtration step.

The sonicated solution is passed through a membrane filter with an appropriate pore size (e.g., 0.45 μm or smaller), effectively removing large, extraneous particles while retaining the nanosized and solubilized bioactive fraction. This step ensures the resulting solution exhibits improved uniformity in particle size distribution and is suitable for efficient SLN encapsulation.

An example of the manufacturing process of whole Spirulina SLNs is disclosed below. In accordance with an aspect of the present invention, palmitic acid is dissolved in 100% ethanol (1.5 mg/mL) and heated to 70° C. Natural and sustainable ingredients that can be used as alternatives to palmitic acid, include stearic acid, behenic acid, lauric acid, cetyl alcohol, cocoa butter, shea butter, carnauba wax, candelilla wax, beeswax, and glycerol monostearate (GMS). Other natural lipids, such as oleic acid, linoleic acid, alpha-linolenic acid, gamma-linolenic acid, triglycerides such as medium-chain triglycerides (MCTs) or long-chain triglycerides (LCTs), and natural oils such as coconut oil, olive oil, and sunflower oil. Additional naturally derived lipid components such as 1-monolaurin, phosphatidylcholine, and phosphatidylethanolamine may also be used. Nanosized Spirulina in dH₂O (distilled water) is heated to 70° C. and combined with the palmitic acid solution at 20% w/w. This lipid-Spirulina mixture is stirred at 70° C. for 5 minutes. While elevated temperatures can degrade proteins, phycocyanin (a key component of Spirulina), degradation at 70° C. is minimal. Phycocyanin degradation is only about 17.40±0.05% phycocyanin degradation when microencapsulated Spirulina platensis is exposed to 100° C. for 30 minutes. Since some embodiments in accordance with the present invention use 70° C. for only 5 minutes, phycocyanin retention greater than 83% is possible. In some embodiments in accordance with the present invention, a 10% surfactant solution is made with Poloxamer-407 dissolved in 2 mL dH₂O and heated to 45° C. While Poloxamer-407 is a synthetic surfactant that is generally regarded as safe and is approved by the US FDA for pharmaceutical applications, some patients prefer natural surfactants due to concerns about ingredients that could exacerbate their symptoms or trigger flares. Natural and sustainable replacements for Poloxamer-407 include lecithin, saponins, Quillaja extract, gum arabic, chitosan, pectin, xanthan gum, alginate, soy protein isolate, rice bran extract, alkyl glycoside (e.g., decyl glucoside), carrageenan, cholesterol, lanolin, or phytosterol. The lipid-Spirulina mixture and the surfactant solution are promptly mixed and stirred at 70° C. for 5 minutes. The combined solution is then cup-sonicated at 45% amplitude (43° C. for 10 minutes) before being cooled to room temperature for 35 minutes. The Spirulina SLNs are quantified using dynamic light scattering (DLS) to measure the mean particle, PDI, and zeta potential. Spirulina SLNs can be fabricated with a mean particle size of less than 200 nm using two 10-minute sonications.

In another embodiment, Spirulina SLNs can be manufactured by dissolving palmitic acid in 100% ethanol (1.5 mg/mL), heating it to 70° C., heating nanosized Spirulina in dH₂O to 70° C., combining it with the palmitic acid solution at 20% w/w, and stirring this lipid-Spirulina mixture at 70° C. for 5 minutes. A 10% surfactant solution is then provided, made with Poloxamer-407 dissolved in 2 mL dH₂O and heated to 45° C. The lipid-Spirulina mixture and the surfactant solution are promptly mixed and stirred at 70° C. for 5 minutes. The combined solution is then cup-sonicated at 45% amplitude (43° C. for 10 minutes) before being cooled to room temperature for 35 minutes.

The interaction of lipid concentration, surfactant percentage, and homogenization speed (intensity) on lipid nanocarrier properties to account for particle size, loading capacity, release kinetics, and aggregation are systematically tested by using methods to optimize SLN formulation. Alternatively, other release kinetics; naturally occurring lipids suitable for SLN loading, such as 1-monolaurin, phosphatidylcholine, or phosphatidylethanolamine (the most abundant phosphatides found in plants and animals) can be used. Similarly, an alternative natural surfactant (alkyl glycoside, carrageenan, cholesterol, lanolin, lecithin, or phytosterol) can be selected.

In some embodiments, sonication is conducted in the presence of nitrogen or argon to prevent oxidation, ensure uniformity, and maintain the bioactivity of compounds. This step is beneficial for compounds that are susceptible to oxidation. Examples of such compounds are alcohols, aldehydes, amines, alkenes, and certain amino acids. Additionally, degassing solvents by removing dissolved oxygen through vacuum degassing or inert gas sparging can significantly reduce oxidation. The addition of antioxidants acts as free radical scavengers, protecting bioactive compounds from oxidative damage. The addition of antioxidants in the fabrication of SLNs serves as a key strategy to protect bioactive compounds from oxidative damage by neutralizing free radicals that may form during sonication, heating, processing, and storage. Various lipid-soluble, water-soluble, and naturally derived antioxidants may be incorporated into SLN formulations to enhance stability, bioactivity retention, and shelf life. Lipid-phase antioxidants, such as tocopherols, carotenoids, and lipid-soluble polyphenols, can prevent peroxidation of encapsulated bioactives. Water-soluble antioxidants, such as ascorbates, thiol-containing compounds, and polyphenolic antioxidants, help mitigate oxidative stress in aqueous environments, thereby protecting hydrophilic bioactives such as proteins, peptides, and polysaccharides. Other antioxidants derived from plant extracts, microbial metabolites, or synthetic sources can provide oxidative protection while also enhancing the therapeutic potential of the SLN formulation.

During SLN fabrication, antioxidants may be co-encapsulated with bioactives, incorporated into the lipid phase, or introduced into the aqueous phase to ensure oxidation resistance throughout processing and storage. The incorporation of antioxidants in SLNs improves encapsulation efficiency, preserves bioactivity, and prevents oxidation-induced structural degradation. Furthermore, antioxidants contribute to enhanced bioavailability, controlled release kinetics, and increased formulation stability. These properties make antioxidant-containing SLNs suitable for pharmaceutical, nutraceutical, cosmetic, and functional food applications. The methods described herein include current approaches or any equivalent method known in the field or developed in the future, ensuring adaptability to advancements in antioxidant stabilization techniques and SLN fabrication processes.

Another key approach is pH control, as some compounds exhibit greater oxidative resistance at specific pH levels, reducing the likelihood of degradation during sonication. By combining these protective measures, oxidation during SLN fabrication can be minimized, ensuring the stability, bioactivity, and therapeutic efficacy of encapsulated compounds.

SLNs undergo drying through lyophilization (freeze-drying), as it effectively preserves the bioactivity of temperature- and oxidation-sensitive compounds while maintaining nanoparticle stability. In this process, the SLN suspension is first mixed with cryoprotectants such as sucrose, trehalose, or mannitol, which prevent nanoparticle aggregation and maintain lipid structure integrity during freezing and sublimation. The ideal cryoprotectant concentration typically ranges between 2-10% (w/v), depending on the formulation's sensitivity to freezing. The SLN dispersion is then placed into suitable freeze-drying containers, such as vials, trays, or bulk containers, and gradually cooled to temperatures between −40° C. and −80° C. in a controlled-rate freezer. Slow freezing at approximately 1° C. per minute minimizes ice crystal formation and structural damage, while rapid freezing at around 10° C. per minute may be used for formulations prone to phase separation.

Once frozen, the SLNs undergo primary drying (sublimation), during which the samples are subjected to vacuum pressure of 10-100 mTorr, while the shelf temperature is gradually increased to −30° C. to −10° C. to initiate the sublimation of ice. This phase typically lasts between 10 to 48 hours, depending on sample volume and equipment efficiency. A controlled sublimation process is critical to preventing lipid destabilization and ensuring bioactive compound stability. Following this step, secondary drying (desorption of bound water) is performed by raising the shelf temperature to 20° C. to 40° C. under continued vacuum conditions to remove residual bound water. This step enhances the long-term stability of the SLNs by reducing moisture content to below 1-3%, minimizing the risk of hydrolysis and degradation. Given the sensitivity of some bioactive compounds such as Spirulina, phycocyanin, or vitamin D3 to oxidation, the dried SLN powder may be further protected by an inert gas flush, such as nitrogen or argon, to extend shelf life and preserve bioavailability.

Once dried, the SLN powder is collected and stored in airtight, light-protected containers at temperatures between 4° C. and 25° C. to prevent degradation due to humidity, oxygen, and UV exposure. To further improve stability, vacuum-sealed packaging or nitrogen flushing is recommended.

Alternative drying methods may also be used depending on the specific manufacturing needs, production scale, and cost considerations. These may include spray drying, which is more cost-effective and scalable but requires stabilizers to prevent thermal degradation, and vacuum drying, which operates at lower temperatures to minimize heat exposure. Supercritical fluid drying is another viable method, particularly for solvent-free processing, while spray freeze-drying offers a hybrid approach that balances efficiency with stability. Additionally, emerging and yet-to-be-discovered drying technologies may further optimize the preservation and scalability of SLNs.

### Dissolvable Microneedles (DMNs)

Dissolvable microneedles are arrays of microscopic needles composed of biodegradable, water-soluble materials. When applied to the skin, the microneedles painlessly pierce the stratum corneum, creating microchannels through which encapsulated nutrients are delivered into the dermis. The microneedles then dissolve, releasing their payload directly into the interstitial fluid for transdermal or systemic absorption. DMNs offer several advantages over traditional transdermal patches, including their ability to bypass the passive diffusion mechanism, deliver larger molecules (>500 Da), and provide sustained release of active ingredients. DMNs may be used to deliver SLNs; this improves the absorption of the bioactive compound within the SLN over traditional transdermal patches.

FIG. 5 shows a diagram of a DMN system delivering SLNs into a patient's skin. The DMN system penetrates the outer layer of the skin (stratum corneum) and the solid lipid nanoparticles (SLNs) and other bioactive compounds are released into the skin's interstitial space. Complete dissolution of the microneedles occurs within 5 to 30 minutes, depending on the specific composition and environmental conditions, leaving no residue or needle material on the skin.

In some embodiments in accordance with the present invention, the dissolvable microneedle based transdermal system is biodegradable.

Biodegradable materials to make the microneedle patches include those derived from natural sources such as hyaluronic acid, chitosan, alginate, gelatin, or carboxymethyl cellulose, which can safely break down in biological environments without leaving harmful residues.

Biodegradable materials for microneedle patches can include a wide range of natural, synthetic, and hybrid polymers, ensuring safe breakdown in biological environments without leaving harmful residues. Natural polymers commonly used in microneedle formulations include hyaluronic acid, chitosan, alginate, gelatin, carboxymethyl cellulose, pullulan, dextran, collagen, pectin, fibrin, and silk fibroin, which provide biocompatibility, controlled degradation, and enhanced stability for drug or nutrient delivery. These materials may be used individually or in combination to optimize microneedle performance.

In addition to naturally derived materials, synthetic biodegradable polymers offer tunable degradation rates and mechanical properties. These include polyvinyl alcohol (PVA), poly(lactic-co-glycolic acid) (PLGA), polylactic acid (PLA), polycaprolactone (PCL), poly(ethylene glycol) (PEG)-based copolymers, and poly(γ-glutamic acid) (PGA). These materials degrade into non-toxic byproducts and may be tailored for rapid dissolution or sustained release, depending on the application.

To further enhance mechanical strength, stability, or bioactivity, crosslinking agents such as genipin, tannic acid, glutaraldehyde, and citric acid may be incorporated. Additionally, inorganic nanoparticles, including silica, calcium phosphates, hydroxyapatite, and bioactive glass, can be used as reinforcements while SLNs, nanocellulose, or liposome-embedded biopolymers may be utilized.

This list is not exhaustive, and future advancements may lead to the development of novel biodegradable materials, hybrid polymers, or bioengineered biopolymers with enhanced mechanical properties, dissolution profiles, and bioactive delivery capabilities. Any material capable of biodegrading safely within biological environments without leaving toxic residues may be considered for microneedle fabrication, including those yet to be discovered or developed.

### Applications of SLNs

SLN's are especially useful for patients suffering from Crohn's Disease or ulcerative colitis. Crohn's Disease is a type of inflammatory bowel disease (IBD) that causes inflammation in the digestive tract, which may lead to symptoms of diarrhea, abdominal pain, nausea, fever, loss of appetite, weight loss, and fatigue. Currently, it affects over 1 million people in the US and 2.3 million globally. The disease substantially burdens healthcare systems and diminishes people's quality of life. Crohn's Disease causes chronic gastrointestinal inflammation, which can lead to severe complications, such as strictures, fistulas, and abscesses, which often require surgery or hospitalization. Current Crohn's Disease treatments, including oral immunosuppressants, intravenous or subcutaneous biologics, and surgery, have significant side effects and may have high costs.

An anti-inflammatory natural compound that may help patients with Crohn's Disease and other inflammatory conditions is C-Phycocyanin (C-PC), a major active compound in Spirulina. The anti-inflammatory function of C-PC has been extensively investigated; for example, oral administration of C-PC significantly reduced intestine inflammation and colitis in animal models. C-PC also shows promising efficacy in modulating organ inflammation beyond the digestive system. In animal models, C-PC, delivered via nasal spray or oral administration, showed the potential to reduce inflammation in diseases of the liver, lungs, and brain. However, due to its poor stability and susceptibility to protease degradation, its application in the pharmaceutical industry has been restricted. C-PC is sensitive to pH, temperature, and light, so it can be very easily inactivated during storage. Moreover, oral administration of C-PC may not be the best route of administration for treating Crohn's Disease because patients experience gut inflammation, which reduces the absorption of nutrients from the small intestine.

SLNs can solve both the problem of preserving the bioactivity of C-PC during storage and the problem of absorption. Since the C-PC is encapsulated within a lipid matrix, it is protected from degradation, making it easier to use it in multiple application s (e.g., pharmaceuticals, nutraceuticals, cosmetics, food and beverage, veterinary, agricultural). Furthermore, it is possible to deliver SLNs transdermally using a DMN patch, bypassing the digestive system altogether.

SLNs are also useful for providing bioactive proteins and peptides, which play crucial roles in skin repair, wound healing, anti-aging, and muscle regeneration. Thus, SLNs could be incorporated in skin creams, salves, and ointments. Bioactive proteins and peptides play crucial roles in skin repair, wound healing, anti-aging, and muscle regeneration. These compounds are prone to degradation in conventional formulations, but encapsulation in SLNs ensures stability and enhances their therapeutic potential. Examples of bioactive proteins and peptides that can be encapsulated in SLNs include collagen peptides, which support skin repair, wound healing, and anti-aging applications; elastin, which enhances skin elasticity and prevents wrinkle formation; keratin, which strengthens hair, nails, and skin; and fibrin, which is used for wound healing and tissue regeneration.

Additionally, bioactive peptides such as Spirulina-derived peptides offer antioxidant, anti-inflammatory, and immune-modulating properties. Fibronectin aids in cell adhesion and wound healing, while laminin supports neural and tissue regeneration. Myosin and actin assist in muscle repair and regeneration and improve cellular structure and movement. Hemoglobin, when encapsulated in SLNs, enhances oxygen delivery and tissue repair.

SLNs could be administered directly to the skin via ointments, salves, or creams or transdermally to aid in wound healing and anti-aging skin applications.

In an embodiment, SLNs could be administered for ophthalmic applications, where encapsulation of phycocyanin could provide anti-inflammatory or soothing effects for eye conditions, such as dry eye. Other bioactive compounds could also be encapsulated in SLNs for ophthalmic applications.

SLNs can enable controlled release of bioactive peptides and proteins, which is beneficial for wound healing and tissue repair. Additionally, SLNs incorporated into transdermal systems can provide localized anti-inflammatory treatment for joint disorders and pain relief. SLNs also improve the absorption of important micronutrients, such as Vitamin B12, iron, magnesium, zinc, and Coenzyme Q10, especially for individuals with malabsorption disorders. Furthermore, SLNs can facilitate the controlled release of wound-healing compounds, offering a targeted approach for post-surgical, chronic wound care, and diabetic foot care. Pharmaceutical companies can utilize SLNs to enhance the stability and delivery of small synthetic compounds, improving the overall efficacy of these therapeutic agents.

In the cosmetic and dermatological sectors, SLN technology provides multiple applications for skin health and repair. SLN-encapsulated phycocyanin and Coenzyme Q10 offer protection against oxidative stress and environmental damage, making them useful in anti-aging and skin repair formulations. SLNs containing collagen, peptides, and phycocyanin can enhance wound healing and reduce scars, thereby supporting skin regeneration. SLNs can also be used in hair loss treatments by encapsulating bioactives that stimulate hair growth in products such as shampoos, serums, and scalp treatments.

SLNs improve the transdermal absorption of essential vitamins and minerals, such as Vitamin D, zinc, and magnesium, in topical formulations. Additionally, SLN-based formulations can deliver antioxidants and provide hydration for lip protection, which is beneficial in soothing lip balms. SLN formulations are also effective in under-eye treatments, where they help reduce puffiness, inflammation, and oxidative damage in eye creams and serums. In post-surgical skin therapy, SLNs incorporated into dissolvable microneedle systems enhance the penetration of peptides and collagen, promoting skin recovery.

For food and beverage applications, SLNs offer a solution to stabilize Spirulina-derived compounds, improving their taste, color, bioavailability, and shelf life. SLNs also allow for the incorporation of Spirulina into non-green functional foods like pasta, cookies, and energy drinks, without the intense green color that typically comes with Spirulina. SLN-encapsulated Spirulina or phycocyanin can enhance plant-based proteins in protein bars and meat alternatives, providing additional nutritional benefits. SLNs also improve the absorption of other nutrients such as non-heme iron, Vitamin D, and polyphenols, which are encapsulated for improved absorption in food fortification. SLNs also stabilize water-soluble vitamins and antioxidants in functional beverages, ensuring enhanced bioavailability and stability.

Additionally, SLNs provide specialized nutrient formulations for individuals with conditions like HIV, anorexia, and gastrointestinal disorders, which require enhanced protein and micronutrient delivery. SLNs can be utilized for targeted delivery of bioactive compounds, providing higher bioavailability, prolonged stability, and sustained therapeutic action across various applications.

In certain embodiments, SLNs are formulated for incorporation into animal and livestock feed to enhance the stability, bioavailability, and targeted delivery of nutritional and therapeutic bioactives. These SLNs may encapsulate algae-derived compounds such as Spirulina, C-phycocyanin, fucoxanthin, or astaxanthin, which are known for their anti-inflammatory, antioxidant, and immune-supportive properties. The lipid matrix protects these sensitive bioactives from degradation during feed processing (e.g., pelleting or extrusion) and from gastric conditions, while enabling controlled release in the intestinal tract. The SLNs may also encapsulate additional bioactives such as probiotics, digestive enzymes, fat-soluble vitamins, or phytochemicals to support gut health, weight gain, feed efficiency, and disease resilience in poultry, swine, ruminants, aquaculture species, or companion animals. These SLNs may be incorporated into powdered premixes, feed pellets, mineral licks, oral pastes, or boluses, and are formulated using food-safe lipids and surfactants to ensure biocompatibility and regulatory compliance in animal nutrition.

In another embodiment, the solid lipid nanoparticles (SLNs) are applied in agricultural systems to enhance the delivery, stability, and bioavailability of bioactive compounds for plant health, soil conditioning, or crop protection. SLNs may encapsulate natural or synthetic agroactives such as micronutrients (e.g., zinc, magnesium, iron), algal extracts, phytohormones (e.g., auxins, gibberellins), antifungal agents, biopesticides, or antioxidant compounds to improve plant growth, stress tolerance, and resistance to pathogens. Encapsulation in SLNs protects these sensitive compounds from degradation caused by UV light, temperature fluctuations, and microbial activity in soil or water, while enabling controlled or sustained release at the rhizosphere or foliar surface. SLNs may be applied via foliar spray, fertigation, seed coating, or soil drenching, and are particularly useful in delivering hydrophobic or unstable bioactives in aqueous-based agricultural systems. The use of biodegradable lipids and natural surfactants ensures environmental compatibility, reduced runoff, and improved uptake efficiency, offering a sustainable and precision-targeted approach to plant nutrition and protection.

In another embodiment, the solid lipid nanoparticles (SLNs) are formulated for use in veterinary medicine to improve the delivery, stability, and bioavailability of therapeutic and nutritional agents for animals. SLNs may encapsulate bioactive compounds such as Spirulina-derived nutrients, C-phycocyanin, essential fatty acids, vitamins, antimicrobials, anti-inflammatory agents, or immunomodulators, and may be administered to companion animals, livestock, or aquaculture species. Encapsulation within SLNs offers protection against enzymatic degradation and oxidation, enables controlled or sustained release, and enhances oral, transdermal, injectable, or mucosal absorption. In veterinary contexts, SLNs may improve therapeutic compliance by enabling palatable oral formulations, odor-masked topicals, or transdermal delivery systems for long-acting delivery. In livestock, veterinary SLNs may be integrated into feed, oral pastes, boluses, or injectables to support disease prevention, weight gain, reproductive health, and stress recovery. The use of biocompatible lipids and natural surfactants allows for safe administration across species and enables precision veterinary formulations tailored to species-specific metabolic rates and nutrient requirements.

By leveraging SLNs for controlled and targeted delivery, this technology offers high bioavailability, prolonged stability, and sustained therapeutic action across multiple industries. The disclosed innovation overcomes key limitations of conventional delivery systems, making it a transformative advancement in the pharmaceutical, nutraceutical, cosmetic, food and beverage, and transdermal industries as well as for veterinary and agricultural use.

### Experimental Data

Nanosizing Spirulina: Spirulina powder (Arthrospira platensis) was dissolved in distilled water at three concentrations: 10 mg/mL, 50 mg/mL, and 100 mg/mL. The mean particle diameter was measured three times using dynamic light scattering (DLS) before and after cup-sonication in 15 second intervals (35% maximum sonication amplitude). Mean particle diameter can be calculated using the Intensity and Number weighting models in the DLS software (Kalliope, Anton Paar). The Intensity model calculates mean diameter of nanoparticles as a function of the intensity of light scattered, and a few larger particles in a heterogeneous solution will highly influence mean diameter. The Number model, however, calculates the nanoparticle diameter based upon the absolute number of particles participating in the scattering of light. The Number weighting model is a better reflection of nanoparticle diameter in a homogenous solution and therefore was used for nanoparticle diameter in this experiment. For the 10 mg/mL Spirulina concentration, sonication successfully reduced nanosized Spirulina particles to 43.76±21.06 nm. Prior to cup-sonication, mean particle diameter was 529.25±52.05 nm (FIG. 6). After four 15-second sonications, Spirulina particle size consistently decreased below the target of 200 nm. Longer sonication times further reduced particle size.

The unsonicated Spirulina solution had a PDI of 0.07±0.22, below the threshold of 0.30 after 30 seconds of vortexing at high speed. Following each of the four 15-second sonications, the PDI remained below 0.30 (0.25±0.82; 0.25±0.22; 0.25±0.19; 0.27±0.12, respectively) with no significant difference between groups (P=0.36). The zeta potential of the nano-sized Spirulina was-22.27±0.43 mV. While the absolute value of the zeta potential of the nano-sized Spirulina is less than the target of 25 mV, the zeta potential may be further decreased by sonicating the Spirulina in macromolecular substances such as albumin, Dextran, or polyethylene glycol.

Spirulina SLNs: To ensure a homogenous Spirulina solution with a mean diameter <200 nm, we first nano-sized Spirulina powder as described above prior to SLN fabrication. We then employed a widely accepted modified melt-ultrasonication method to make SLNs. The mean diameter of the Spirulina SLN solution (palmitic acid, Spirulina, and surfactant) was relatively unaffected by 10-minute sonication, but a second 10-minute sonication after cooling significantly reduced SLN diameter to 54.02±20.24 nm (P=0.01). The PDI of the SLN solution was within the acceptable threshold of 0.30, reaching 0.29±0.01 after sonication. Finally, the zeta potential of the nanosized Spirulina before encapsulation was −22.13±0.38 mV, below the stability threshold of −25 mV for transdermal diffusion. However, after sonication, the Spirulina SLNs showed a mean zeta potential of −29.87±4.64 mV, exceeding the absolute zeta potential of 25 mV. This indicates that our SLN fabrication process produces stable SLNs with sufficient surface charge to prevent agglomeration.

Upon encapsulation into SLNs, the visual color of the Spirulina changed from its original deep green to an off-white/yellowish hue, indicating effective lipid encapsulation and pigment shielding. This color transformation enhances its applicability in cosmetics, functional foods and beverages, and nutraceuticals, where color neutrality is preferred. Additionally, while raw Spirulina has a characteristic marine odor, the SLN formulation significantly reduces this odor due to the lipid encapsulation process, making it more suitable for consumer applications where odor neutrality is required.

Beyond its rich antioxidant and bioactive properties, Spirulina contains all nine essential amino acids, making it an ideal plant-based protein replacement and supplement for food and beverage applications. The SLN encapsulation improves dispersibility and stability, allowing for seamless integration into protein shakes, dairy alternatives, nutritional bars, and other functional foods while eliminating the green color and strong marine taste that typically limit Spirulina's commercial use.

Furthermore, Spirulina SLNs can be effectively utilized as a natural and sustainable skincare ingredient, where nanoencapsulation enhances penetration into the deeper dermal layers to deliver essential vitamins, minerals, antioxidants, and bioactive nutrients directly to skin cells. The nanoencapsulation of skincare ingredients into SLNs allows for improved skin nourishment, hydration, and cellular rejuvenation, making it a high-value ingredient for anti-aging, wound healing, and restorative skincare formulations. The bioavailability of encapsulated Spirulina-derived compounds is significantly enhanced, ensuring controlled release and prolonged therapeutic effects in cosmetic applications.

These data demonstrate the successful fabrication of Spirulina encapsulated SLNs with the desired diameter that meet the PDI and zeta potential criteria for optimal transdermal or oral delivery.

In an embodiment, the fabrication of C-Phycocyanin (C-PC)-loaded Solid Lipid Nanoparticles (SLNs) is performed using a modified melt-ultrasonication method to optimize stability, encapsulation efficiency, and bioavailability for transdermal delivery. The process begins with the selection of a solid lipid, wherein palmitic acid is dissolved in 100% ethanol and heated to 70° C. to ensure complete melting. In parallel, C-PC is dissolved in distilled water at 0.5 mg/mL and heated to 70° C., allowing for temperature equilibration between phases. The surfactant Poloxamer-407 (2.5% w/w) is incorporated into the aqueous phase to enhance stability and dispersion. The lipid phase is subsequently introduced into the aqueous surfactant and C-PC solution under continuous stirring at 70° C. for 5 minutes to promote uniform mixing.

To achieve nanoscale encapsulation, the formulation undergoes ultrasonication using a cup sonicator at 50% amplitude for 3.75 minutes in a pulsed manner (4 sec on, 1 sec off) at 45° C. This sonication step facilitates nanoparticle size reduction, improves emulsification, and ensures homogeneous dispersion of C-PC within the lipid matrix. Immediately following ultrasonication, the solution is rapidly cooled at 4° C. for 30 minutes while stirring, inducing lipid solidification and preventing nanoparticle aggregation. The resulting SLNs are analyzed to confirm that they meet predefined stability and transdermal absorption criteria. Dynamic light scattering (DLS) is used to measure the mean particle size, with a target diameter of ≤350 nm to ensure effective penetration through the skin. The polydispersity index (PDI) is maintained below 0.30, ensuring a uniform size distribution that minimizes aggregation risks. The zeta potential is evaluated to confirm stability, with an absolute value of >25 mV, ensuring sufficient electrostatic repulsion between particles to prevent agglomeration.

Encapsulation efficiency (EE) is determined by centrifuging the SLN solution at 14,500 RPM for 45 minutes to separate unencapsulated C-PC from the nanoparticles. Amino Acid Analysis (AAA) are used to quantify encapsulated and free C-PC, with results demonstrating a 99.93% encapsulation efficiency (EE), significantly exceeding the target threshold of >50% EE. Minimal C-PC (0.5 μg) was detected in the supernatant, confirming nearly complete entrapment of C-PC within the SLN matrix. Amino acid profiling further verified structural integrity of C-PC within SLNs, indicating that encapsulation does not degrade or alter its bioactivity.

To ensure functional stability, bioactivity retention is evaluated through DPPH free radical scavenging assay, confirming that encapsulated C-PC retains antioxidant capacity. Additionally, anti-inflammatory activity is validated by treating Caco-2 cells stimulated with IL-1ß (10 ng/ml) with C-PC SLNs, followed by qRT-PCR analysis of inflammatory cytokines (IL-6, IL-8, TNF-α). Results confirm that SLN encapsulation preserves C-PC's biological activity, making it a viable formulation for transdermal therapeutic applications.

In alternative embodiments, process modifications may be employed to optimize nanoparticle characteristics, including reducing lipid viscosity by substituting palmitic acid with lauric acid (44° C. melting point), increasing surfactant concentration to lower interfacial tension, extending ultrasonication duration or amplitude to enhance cavitation, or employing rapid cooling at 2° C. post-sonication to improve nanoparticle formation. The melt-ultrasonication method described herein provides a scalable and reproducible approach for nanoencapsulation of natural bioactive compounds, enabling enhanced stability, bioavailability, and controlled release for pharmaceutical, nutraceutical, and cosmetic applications.

In an embodiment, vitamin D3 (cholecalciferol) is encapsulated into solid lipid nanoparticles (SLNs) using a modified melt-ultrasonication method to enhance transdermal absorption, stability, and bioavailability. This process optimizes particle size (<200 nm), polydispersity index (PDI <0.30), zeta potential (>25 mV, absolute value), and encapsulation efficiency (>50%), ensuring effective transdermal delivery. The encapsulation process begins with the selection of lipophilic carriers and surfactants to ensure stability and uniform dispersion. Vitamin D3 powder (Sigma Aldrich) is dissolved in ethanol at 10 mg/mL, 50 mg/mL, and 100 mg/mL concentrations to determine optimal encapsulation conditions. The mean particle size of unencapsulated vitamin D3 is measured using Dynamic Light Scattering (DLS), with an initial diameter of 124.6±0.1 nm and a PDI of 10.17±2.26 before SLN fabrication. Palmitic acid (1.5 mg/mL) is dissolved in 99.9% ethanol and heated to 70° C. to ensure complete melting and homogenization, while Poloxamer-407 (1% w/w) is dissolved in distilled water and heated to 45° C. to facilitate dispersion. The vitamin D3 ethanol solution is combined with the lipid phase at 20% w/w, followed by mixing with the surfactant solution under continuous stirring at 70° C. for 5 minutes to ensure uniform dispersion.

The combined lipid-vitamin D3 emulsion undergoes ultrasonication to ensure nano-sized SLN formation, promoting stability and uniformity. Cup-sonication is performed at 45% amplitude (45° C. for 10 minutes) in pulse mode (4 sec on, 1 sec off) to break down lipid droplets and encapsulate vitamin D3 efficiently. After sonication, three cooling methods are tested to evaluate their impact on SLN properties: Rapid Cooling (RC, 2° C. for 30 minutes, stirring continuously), Intermittent Cooling (IC, 15° C. cooling rate), and Slow Cooling (SC, 23° C., room temperature cooling). The fabricated vitamin D3 SLNs are evaluated for size, uniformity, and stability using DLS and Liquid Chromatography-Mass Spectrometry (LC-MS). The mean particle size for all SLN formulations remains below 200 nm, ensuring optimal transdermal absorption, with RC producing 82.8±8.4 nm, IC at 96.1±30.6 nm, and SC at 69.8±9.2 nm. The polydispersity index (PDI) values of RC: 0.29±0.05, IC: 0.32±0.02, and SC: 0.36±0.07 confirm that rapid cooling produces the most uniform SLN size distribution, ensuring stability. The zeta potential values (RC: −30.13±2.08 mV, IC: −25.22±0.26 mV, SC: −5.34±0.15 mV) demonstrate that rapid cooling results in the most stable SLN formulation, exceeding the absolute zeta potential threshold of +25 mV.

Encapsulation efficiency (EE) is determined by centrifuging the SLN suspension at 14,500 RPM for 45 minutes, and the supernatant is analyzed using LC-MS to quantify unencapsulated vitamin D3.

In an alternative embodiment, a natural surfactant (decyl glucoside) replaces Poloxamer-407 to create a fully biodegradable and naturally derived SLN formulation. Decyl glucoside, a plant-based surfactant, has been validated in prior studies for encapsulating lipid-soluble compounds and is tested under the same conditions as the Poloxamer-407 formulation, with SLNs analyzed for size, PDI, zeta potential, and EE to confirm compatibility with the modified surfactant system.

This vitamin D3 SLN nanoencapsulation protocol successfully produces stable nanoparticles with optimal size, distribution, and encapsulation efficiency, ensuring effective transdermal absorption and controlled vitamin D3 release. The rapid cooling method results in the most stable SLNs, with high zeta potential and minimal aggregation, making these formulations ideal for integration into a dissolvable microneedle (DMN) patch for transdermal nutrient supplementation applications.

1. A solid lipid nanoparticle, comprising:
a. a lipid matrix comprising at least one biocompatible lipid forming a generally spherical particle;
b. a surfactant layer on the exterior of the particle, comprising at least one surfactant;
c. at least one bioactive compound dispersed within the lipid matrix, wherein the bioactive compound is at least one of a group comprising a vitamin, mineral, enzyme, algae-derived material, protein, peptide, amino acid, antioxidant, synthetic small molecule, plant-derived volatile compound, a botanical extract, a polyphenol, or a naturally derived phytochemical compound;
d. wherein the solid lipid nanoparticle has at least one of the following: diameter of 10 nm to approximately 1000 nm, a PDI of less than 0.30, and a zeta potential (absolute value) of at least 25 mV.

2. The solid lipid nanoparticle of claim 1, wherein the lipid matrix comprises at least two distinct biocompatible lipids.

3. The solid lipid nanoparticle of claim 1, wherein the lipid matrix comprises exclusively natural lipids.

4. The solid lipid nanoparticle of claim 1, wherein the surfactant layer comprises at least two distinct surfactants.

5. The solid lipid nanoparticle of claim 1, wherein the surfactant layer comprises exclusively natural surfactants.

6. The solid lipid nanoparticle of claim 1, wherein the bioactive compound is an algae-derived material.

7. The solid lipid nanoparticle of claim 6, wherein the bioactive compound is Spirulina.

8. The solid lipid nanoparticle of claim 6, wherein the bioactive compound is C-phycocyanin.

9. The solid lipid nanoparticle of claim 1, wherein the bioactive compound is a psychedelic or neuroactive compound selected from psilocybin, DMT, 5-MeO-DMT, Salvinorin A.

10. The solid lipid nanoparticle of claim 1, wherein the bioactive compound is derived from mushrooms.

11. The solid lipid nanoparticle of claim 1, further comprising:
a. a second bioactive compound distinct from the bioactive compound, wherein a mixture of the bioactive compound and the second bioactive compound is encapsulated within the lipid matrix.

12. A method of making a solid lipid nanoparticle composition, comprising:
a. dissolving at least one bioactive compound in a first solvent to create a bioactive phase,
i. wherein the bioactive compound is selected from a group comprising a vitamin, mineral, enzyme, algae-derived material, protein, peptide, amino acid, antioxidant, synthetic small molecule, naturally derived phytochemical compound, botanical extract, and polyphenol;
ii. wherein the first solvent is selected from a group comprising distilled water, ethanol, or pharmaceutically acceptable solvent;
b. dissolving at least one lipid in a second solvent to create a lipid phase,
i. wherein at least one lipid is selected from a group comprising natural lipids, synthetic lipids, and semi-synthetic lipids;
ii. wherein the second solvent is selected from a group comprising ethanol or a pharmaceutically acceptable solvent;
c. heating the lipid phase to a temperature at or above a melting point of at least one lipid;
d. adding at least one surfactant to water to create a surfactant solution, wherein the surfactant is selected from a group comprising natural surfactants, non-ionic surfactants, anionic surfactants, cationic surfactants;
e. mixing the lipid phase, bioactive phase, and surfactant solution to create a mixture;
f. heating the mixture to a temperature sufficient to fully melt the lipid and allow dispersion of the bioactive compound for a duration of at least 1 minute;
g. inputting mechanical energy to the mixture;
h. cooling the mixture under conditions sufficient to solidify the lipid;
i. drying the solid lipid nanoparticles.

13. The method of claim 12, wherein the step of inputting mechanical energy to the mixture comprises sonicating the mixture at a frequency between 1-100 W, 10-60% amplitude for 1 to 15 minutes.

14. The method of claim 12, wherein the step of drying the solid lipid nanoparticles comprises at least one of the following:
a. mixing the mixture with at least one cryoprotectant at a concentration ranging from 2-10% (w/w);
b. placing the mixture into at least one container that can withstand freeze-drying temperatures;
c. gradually cooling the mixture to a temperature ranging between −40° C. and −80° C.;
d. sublimating the mixture by subjecting the mixture to vacuum pressure of 10-100 mTorr while increasing the temperature to −30° C. to −10° C. to initiate the sublimation of ice, for a time period between 10 and 48 hours;
e. secondary drying the mixture by raising the temperature to 20° C. to 40° C. under continued vacuum conditions to remove residual bound water.

15. The method of claim 12, wherein the step of gradually cooling the mixture is performed at approximately 1° C. per minute.

16. The method of claim 12, wherein the step of gradually cooling the mixture is performed at approximately 10° C. per minute.

17. The method of claim 12, wherein the step of drying the solid lipid nanoparticles comprises spray drying.

18. The method of claim 12, wherein the step of drying the solid lipid nanoparticles comprises vacuum drying.

19. The method of claim 12, wherein the step of drying the solid lipid nanoparticles comprises supercritical fluid drying.

20. The method of claim 12, wherein the step of drying the solid lipid nanoparticles comprises spray freeze-drying.

21. The method of claim 12, further comprising:
a. after dissolving a bioactive compound in a first solvent to create the bioactive phase, sonicating the bioactive phase until its mean particle size is 100 nm or less.

22. The method of claim 21, wherein the bioactive compound is whole spirulina.

23. The method of claim 21, wherein the bioactive compound is C-Phycocyanin.

24. The method of claim 12, wherein the at least one lipid is a natural lipid.

25. The method of claim 12, wherein the concentration of bioactive ingredient in the bioactive phase is 5-50 mg/mL.

26. The method of claim 12, wherein the concentration of lipid in the lipid phase is 1-20% w/w.

27. The method of claim 12, wherein the surfactant is a natural surfactant.

28. A system for delivering a bioactive compound to a living being, comprising:
a. a transdermal delivery system comprising at least one dissolvable microneedle, wherein the at least one dissolvable microneedle comprises a plurality of solid lipid nanoparticles.

29. A system for delivering a bioactive compound to a living being, comprising at least one of the following:
a. a tropical formulation comprising a plurality of solid lipid nanoparticles of claim 1;
b. a food item comprising a plurality of solid lipid nanoparticles of claim 1;
c. a beverage comprising a plurality of solid lipid nanoparticles of claim 1;
d. a fabric comprising a plurality of solid lipid nanoparticles of claim 1;
e. an ophthalmic drop comprising a plurality of solid lipid nanoparticles of claim 1;
f. an oral medication comprising a plurality of solid lipid nanoparticles of claim 1;
g. a powder comprising a plurality of solid lipid nanoparticles of claim 1;
h. a suspension comprising a plurality of solid lipid nanoparticles of claim 1;
i. an emulsion comprising a plurality of solid lipid nanoparticles of claim 1;
j. a film comprising a plurality of solid lipid nanoparticles of claim 1;
k. a cream comprising a plurality of solid lipid nanoparticles of claim 1;
l. a gel comprising a plurality of solid lipid nanoparticles of claim 1;
m. a skin patch comprising a plurality of solid lipid nanoparticles of claim 1;
n. an edible item comprising a plurality of solid lipid nanoparticles of claim 1;
o. a nasal spray comprising a plurality of solid lipid nanoparticles of claim 1
p. an injectable substance comprising a plurality of solid lipid nanoparticles of claim 1;
q. an inhalable formulation comprising a plurality of solid lipid nanoparticles of claim 1;
r. a suppository comprising a plurality of solid lipid nanoparticles of claim 1;
s. a buccal formulation comprising a plurality of solid lipid nanoparticles of claim 1;
t. a textile product comprising a plurality of solid lipid nanoparticles of claim 1
u. a veterinary medication comprising a plurality of solid lipid nanoparticles of claim 1;
v. an agricultural product comprising a plurality of solid lipid nanoparticles of claim 1.

30. The solid lipid nanoparticle of claim 1, further comprising a targeting component selected from pH-responsive coatings, ligand-conjugated lipids, receptor-targeting peptides, or antibody fragments to enhance tissue-specific delivery.
